(19) United States
(12) Patent Application Publication
(10) Pub. No.: US 2025/0296079 A1
(43) Pub. Date: Sep. 25, 2025

(54) DEVICES, METHODS, AND SYSTEMS FOR MEASURING AND RECORDING A REACTANT ARRAY

(71) Applicant: SENSILL, INC., Concord, CA (US)

(72) Inventor: William Shea, Martinez, CA (US)

(73) Assignee: SENSILL, INC., Concord, CA (US)

(21) Appl. No.: 19/229,750

(22) Filed: Jun. 5, 2025

Related U.S. Application Data
(63) Continuation of application No. PCT/US2023/083073, filed on Dec. 8, 2023.

(51) Int. Cl. B01L 3/00; G01N 21/25; G01N 21/78

(52) U.S. Cl. CPC B01L 3/502; G01N 21/251; G01N 21/78; B01L 2200/026; B01L 2200/0689; B01L 2300/0663; B01L 2300/0681; B01L 2300/123; B01L 2400/0487; B01L 2400/0633

(57) ABSTRACT

Devices, systems, and methods include a system for analyzing reactant arrays (e.g., reactant arrays of colorimetric sensor arrays). The system may include a cartridge including a reactant array and a device for analyzing the reactant array when the cartridge is received by or in the device. The cartridge may include input port, an output port, and a component between the input port and the output port. The device may be configured to engage the pump component to pump fluid through a pathway in fluid communication with the input port, the output port, and the reactant array. The reactant array may include one or more reactants (e.g., analyte sensitive material).

### CROSS-REFERENCE TO RELATED APPLICATIONS

This application is a continuation of International Application No. PCT/US2023/083073, filed Dec. 8, 2023, which claims priority to: U.S. Provisional Patent Application Ser. No. 63/431,507, filed Dec. 9, 2022, the entirety of which is incorporated herein by reference; U.S. Provisional Patent Application Ser. No. 63/431,510, filed Dec. 9, 2022, the entirety of which is incorporated herein by reference; U.S. Provisional Patent Application Ser. No. 63/431,519, filed Dec. 9, 2022, the entirety of which is incorporated herein by reference; U.S. Provisional Patent Application Ser. No. 63/431,525, filed Dec. 9, 2022, the entirety of which are incorporated herein by reference; U.S. Provisional Patent Application Ser. No. 63/431,528, filed Dec. 9, 2022, the entirety of which are incorporated herein by reference; U.S. Provisional Patent Application Ser. No. 63/431,533, filed Dec. 9, 2022, the entirety of which are incorporated herein by reference.

### TECHNICAL FIELD

The present disclosure pertains to sensing and analysis tools, and the like. More particularly, the present disclosure pertains to devices and systems for sensing and analyzing chemical substances, and methods for manufacturing and using such devices.

### BACKGROUND

A wide variety of devices have been developed for collection, storing, sensing, and analysis of samples. These devices are manufactured by any one of a variety of different manufacturing methods and may be used according to any one of a variety of methods. Of the known medical devices and methods, each has certain advantages and disadvantages.

### BRIEF SUMMARY

This disclosure provides design, material, manufacturing method, and use alternatives for sensing and analysis devices. Although it is noted that collection, storing, sensing, and analysis approaches and systems are known, there exists a need for improvement on those approaches and systems.

An example system may include a cartridge and a device for analyzing the colorimetric sensor array when the cartridge is received in the device, wherein the cartridge may include a colorimetric sensor array, an input port, an output port, and a pathway configured to be in fluid communication with the input port, the output port, and the colorimetric sensor array

Alternatively or additionally to any of the embodiments in this section, the pathway may be configured to be in fluid communication with a target area.

Alternatively or additionally to any of the embodiments in this section, the cartridge and the device may form a closed loop system defining the pathway configured to be in fluid communication with the target area and causing fluid passing over the colorimetric sensor array to return to the target area.

Alternatively or additionally to any of the embodiments in this section, the cartridge may be a single-use component and the device may be a reusable component.

Alternatively or additionally to any of the embodiments in this section, the device for analyzing the colorimetric sensor array may include a pump drive component and the cartridge may include a pump driven component configured to be driven by the pump drive component when the cartridge is positioned within the device for analyzing the colorimetric sensor array.

In another example, a cartridge for use with a device for analyzing a colorimetric sensor array may include a colorimetric sensor array, a pathway extending over the colorimetric sensor array, the pathway is configured to receive fluid from a target area, and a single-use feature configured to prevent the colorimetric sensor array from being reused.

Alternatively or additionally to any of the embodiments in this section, the single-use feature may be an electrical single-use feature.

Alternatively or additionally to any of the embodiments in this section, the single-use feature may be a mechanical single-use feature

Alternatively or additionally to any of the embodiments in this section, the single-use feature may be one or more of the following types of single-use features: a mechanical single-use feature, an electrical single-use feature, an optical single-use feature, a chemical single-use feature, magnetic, single-use feature, and an electromechanical single-use feature.

Alternatively or additionally to any of the embodiments in this section, the colorimetric sensor array may include a substrate.

Alternatively or additionally to any of the embodiments in this section, the substrate may be configured to filter undesirable molecules from the fluid.

Alternatively or additionally to any of the embodiments in this section, the system may further include a filter in the pathway, wherein the filter may be configured to filter undesirable molecules from the fluid.

In another example, a cartridge for use with a device for analyzing a colorimetric sensor array may include a substrate, an array of reactants applied to the substrate, and a pathway extending over the array of reactants, the pathway is configured to receive fluid from a target area, and wherein the substrate may be configured to filter undesirable molecules from the fluid.

In another example, a cartridge for use with a device for analyzing a colorimetric sensor array may include a colorimetric sensor array, a pathway extending over the color sensor array, the pathway is configured to receive fluid from a target area, and a filter in the pathway, the filter is configured to filter undesirable molecules from the fluid.

In another example, system may include a cartridge, the cartridge includes a colorimetric sensor array, an input port, an output port, and a flexible membrane, the cartridge includes a pathway configured to be in fluid communication with the input port, the output port, the colorimetric sensor array, and the flexible membrane, a device for analyzing the colorimetric sensor array when the cartridge is received in the device, and a pump configured to apply a force to the flexible membrane to pump fluid long the pathway and along the colorimetric sensor array.

In another example, a system may include a cartridge, the cartridge includes a reactant array, an input port, an output port, and a pump component between the input port and the output port, a device for analyzing the reactant array when the cartridge is received in the device, wherein the device may be configured to engage the pump component to pump fluid through a pathway in fluid communication with the input port, the output port, and the reactant array.

Alternatively or additionally to any of the embodiments in this section, wherein the pump component may be fluidly isolated from the pathway.

Alternatively or additionally to any of the embodiments in this section, the device may include an actuator configured to actuate the pump component.

Alternatively or additionally to any of the embodiments in this section, the pump component may include an impeller.

Alternatively or additionally to any of the embodiments in this section, the pump component may include a piezoelectric diaphragm configured to electrically connect with the device.

Alternatively or additionally to any of the embodiments in this section, the pump component may include a flexible diaphragm at least partially defining the pathway.

Alternatively or additionally to any of the embodiments in this section, the device may include a driving component configured to engage the flexible diaphragm to pump fluid through the pathway.

Alternatively or additionally to any of the embodiments in this section, the system may further include a first valve and a second valve, wherein the first valve may be configured to open and the second valve may be configured to close in response to a first movement of the flexible diaphragm and the first valve may be configured to close and the second valve may be configured to open in response to a second movement of the flexible diaphragm.

Alternatively or additionally to any of the embodiments in this section, the pathway may be configured to be in fluid communication with a target area and the cartridge forms a closed loop system defining the pathway configured to be in fluid communication with the target area and causing fluid passing to the reactant array to return to the target area.

Alternatively or additionally to any of the embodiments in this section, the cartridge may be a single-use component and the device is a reusable component.

Alternatively or additionally to any of the embodiments in this section, the cartridge may include one or more seals configured to seal the input port and the output port to hermetically seal the reactant array within the cartridge.

Alternatively or additionally to any of the embodiments in this section, the device for analyzing the reactant array may include a pump drive component and the pump component of the cartridge may be a pump driven component configured to be driven by the pump drive component when the cartridge is positioned within the device for analyzing the reactant array.

Alternatively or additionally to any of the embodiments in this section, the cartridge may include a cartridge housing that houses the reactant array and the pump component and the device may include a device housing configured to at least partially receive the cartridge housing.

In another example, a cartridge for use with a device for analyzing reactant arrays may include a reactant array, a pathway extending to the reactant array, the pathway is configured to receive fluid from a target area, and a driven pump component configured to engage a drive pump component of the device for analyzing the reactant array to pump fluid through the pathway and to the reactant array.

Alternatively or additionally to any of the embodiments in this section, the driven pump component may be fluidly isolated from the pathway.

Alternatively or additionally to any of the embodiments in this section, the driven pump component may be selected from a group consisting of a flexible diaphragm, a rotor, a piezoelectric element, an Archimedes screw, and a plunger.

Alternatively or additionally to any of the embodiments in this section, the cartridge may further include a single-use component configured to prevent the reactant array from being reused.

Alternatively or additionally to any of the embodiments in this section, the single-use component may include one or more of the following types of single-use components: a mechanical single-use component, an electrical single-use component, an optical single-use component, a chemical single-use component, magnetic single-use component, and an electromechanical single-use component.

Alternatively or additionally to any of the embodiments in this section, the cartridge may further include a filter in the pathway, the filter may be configured to remove undesirable molecules from the fluid.

Alternatively or additionally to any of the embodiments in this section, the reactant array may include a substrate and an array of reactants applied to the substrate and the substrate may be the filter.

In another example, a system may include a cartridge, the cartridge includes a reactant array, an input port, an output port, and a flexible membrane, the cartridge includes a pathway configured to be in fluid communication with the input port, the output port, the reactant array, and the flexible membrane, a device for analyzing the reactant array when the cartridge is received in the device, and a pump configured to apply a force to the flexible membrane to pump fluid long the pathway and along the reactant array.

Alternatively or additionally to any of the embodiments in this section, the cartridge may include a component of the pump.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic diagram of an illustrative sensing system;

FIG. 2 is a schematic diagram of an illustrative computing system;

FIG. 3 is a schematic diagram of an illustrative sensing system in a hand of a user;

FIG. 4 is a schematic top view of an illustrative sensing system;

FIGS. 5A and 5B are schematic top and side views, respectively, of an illustrative sensing system;

FIG. 6 is a schematic side view of an illustrative sensing system;

FIG. 7 is a schematic side view of an illustrative sensing system;

FIG. 8 is a schematic top view of an illustrative sensing system;

FIG. 9 is a schematic perspective of an illustrative cartridge;

FIG. 10 is a schematic cross-section view of the illustrative cartridge of FIG. 9, with a sample source coupled therewith;

FIG. 11 is a schematic top view of the illustrative cartridge of FIG. 9, with a sample source coupled therewith;

FIG. 12 is a schematic top view of an illustrative cartridge;

FIGS. 13A-13C schematically depict an illustrative technique for pumping fluid through a cartridge;

FIGS. 14A and 14B schematically depict an illustrative technique for pumping fluid through a cartridge;

FIG. 15 is a schematic diagram of an illustrative technique for analyzing a colorimetric sensor array.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

The term “fluid” is inclusive of both liquids and gases.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all embodiments include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Fluids with concentrations of volatile compounds (e.g., volatile organic compounds (VOCs)) and/or gasses, which may or may not be hazardous, may be sensed, analyzed, and/or monitored. Sensing, analyzing, and/or monitoring of fluids with analytes (e.g., non-volatile or volatile compounds, gases, liquids and/or other fluids) may utilize absorption and/or reflectance measurements of reactants exposed to such fluids for any purpose including, but not limited to, diagnostic hazard warning, manufacturing processes or quality control, record keeping, archival purposes, product development, product-consumer matching, etc.

In some cases, VOCs and/or gasses may be present in ambient fluid (e.g., ambient air, etc.) and sensed, analyzed, and/or monitored using reactants for real-time alarms, to treat subjects, or to collect and/or archive data for health records, regulatory compliance records, etc. Further, VOCs and/or gasses exhaled or emitted, excreted, emanated, released, and/or secreted from a subject (e.g., humans, animals other than humans, food, produce, meat, pathogens, bacteria (e.g., good and/or bad bacteria), plants, wounds, ulcers, surgical sites, skin of a subject, mouth of a subject, nasal passages of a subject, sinuses of a subject, rectum area of a subject, vaginal area of a subject, genitals area of a subject, ear canals of a subject, pores of a subject, etc.) may be sensed, analyzed, and/or monitored to assess hazardous, dangerous, or illegal substances in or at the subject or target site, a lung condition of lungs of a subject, a condition of a blood disease, a condition of infections, conditions related to diseases or biological conditions, conditions related to general health, conditions related to food flavors, conditions related to perfumes or smells, and/or other suitable conditions.

The systems discussed herein for sensing, analyzing, and/or monitoring fluids (e.g., for analytes of interest) may be configured to accurately detect and record a colorimetric sensor array (CSA) spectral response to exposure to the fluids. The systems may utilize techniques for non-invasively detecting one or more analytes of interest (e.g., one or more pathogens responsible for specific human skin infections including, but not limited to, skin infections, urinary tract infections (UTIs), vaginitis, wound infections, ulcers, etc., and/or other suitable analytes) from a fluid using a CSA to allow for early detection of and early implementation of protocols to address one or more conditions associated with any sensed analytes of interest. In one example, enhanced classification of one or more analytes using the systems described herein may enable detection and identification of responsible pathogens at the very beginning stages of a dangerous skin infection, which may result in a high level of protection and probability of a favorable outcome for subjects.

An analysis system (e.g., a CSA analysis system) may include an enclosure or cartridge containing a reactant array (e.g., a CSA having a reactant array) and a device for reading or otherwise analyzing the reactant array in the cartridge before, during, and/or after the reactant array is exposed to a fluid. The device for analyzing the reactant array (e.g., a reader device) may be or may include a spectrometer, an image sensor, and/or other suitable image or light collectors/sensors. In some instances, the device may be configured to be a handheld or a benchtop device. Further, the device for analyzing the reactant array may be configured to be simple to use, minimize human error, reduce cross contamination of samples, and reduce human risk of exposure to analytes received at the device.

In operation, the enclosure or cartridge containing a reactant array may be placed into the device for analyzing the reactant array (e.g., via an opening in the device) during and/or through pre and post exposure to fluid for optimal reactant array image/reflectometer analysis. That is, analysis of the reactant array in the cartridge may include analyzing the reactant array prior to exposure to the fluid, during exposure to the fluid, and/or after exposure to the fluid is complete. Once the analysis of the reactant array in the cartridge is complete, the cartridge may be removed from the device for analyzing the reactant array and discarded. Having the cartridge be disposable may prevent cross-contaminations of the CSA and erroneous analyses (e.g., due to the previous fluids persisting in the tubing and reflowing over the reactant array, undesirably flowing onto a human, etc.).

The cartridge may be any suitable component including one or more reactant arrays (e.g., where the reactant arrays may be permanent or removable from the cartridge) and that may be configured to expose the one or more reactant arrays to fluids from a target area or site. In one example, the cartridge may be a flow cell and may have an input port for receiving fluid (e.g., a fluid from a target area or site) to pass over and/or through the one or more reactant arrays and an output port for outputting the fluid passed through and/or over the one or more reactant arrays. As the fluid passes through and/or over the reactant arrays, the fluid may interact with the chemistries of reactants (e.g., color bars, color dots, predetermined patterns, and/or other suitable reactants) of the reactant arrays and the reactants may react to the interaction (e.g., change color in response to the interaction and/or react in one or more other suitable manners). The spectrometer or other suitable light or image sensor may then read or otherwise analyze the reactions of the reactants to the fluid and determine properties of the fluid based on analysis of the reactions.

The CSA including the reactant array may include a membrane or substrate on or to which the reactants are applied to form the reactant array. In some cases, an entirety of or at least a portion of the membrane or substrate may act as a filter to filter out molecules that are not of interest from a flow of fluid to be passed over and/or through the reactants of the CSA to facilitate allowing the molecules of analytes that are of interest to reach the reactants with a concentration suitable for causing the reactants to react to contact with the analytes. The membrane or substrate configured to operate as a filter may be made from any suitable materials including, but not limited to, biopolymer-based filtration materials, adsorbent materials, and/or other suitable materials. In some examples, the membrane may be configured to interact with particular molecules to enhance reactivity of the reactants with the fluid passing through the pathway and/or to alter incoming analytes into different constituents for greater sensitivity. The membrane or substrate may be sized, shaped, and/or configured differently to address filtering out different molecules. Some variables that may be adjusted to filter out undesirable molecules include, but are not limited to, material of the substrate or membrane, pore sizes in the substrate or membrane, pore concentrations in the substrate or member, pore locations in the substrate or membrane, etc.

In addition to or as an alternative to having a membrane or substrate of the CSA be configured to filter molecules from a fluid containing or at least potentially containing analytes of interest, the cartridge of the analysis system may include one or more filters in a fluid path for removing undesirable molecules from the fluid passing through the fluid path and allowing desirable molecules to pass through the filter. In some examples, the filter may be configured to interact with particular molecules to enhance reactivity of the reactants with the fluid passing through the pathway and/or to alter incoming analytes into different constituents for greater sensitivity. The filter may be sized, shaped, and/or configured differently to address filtering out different molecules. Some variables that may be adjusted to configure the filter include, but are not limited to, material of the filter, pore sizes in the filter, pore concentrations in the filter, pore locations in the filter, etc.

To provide fluids to the reactant array and remove fluid from the reactant array, tubing may connect to the ports and/or be integrally formed with the ports of the cartridge (e.g., as part of the cartridge) at respective first ends. The tubing coupled to or integrally formed with the ports may be coupled to a collector of analytes and/or in fluid communication with fluid at or from a target area (e.g., an area of interest where analytes may be located). Additionally or alternatively, specimens including samples from a target area and/or carriers including samples from the target area may be coupled with or inserted into the cartridge to facilitate providing fluids from a sample or target area to the CSA, where the samples may be obtained from a collector, but this is not required. Example collectors of, concentrators for, and/or systems for collecting analytes include, but are not limited to, the collectors discussed in: PCT Patent Application Publication WO 2022/072827 A1, filed on Oct. 1, 2021, and titled DEVICES, METHODS AND SYSTEMS TO COLLECT, STORE, AND ANALYZE CHEMICAL SUBSTANCES, which is hereby incorporated by reference in its entirety for all purposes; PCT Patent Application Publication WO 2022/099021 A1, filed on Nov. 5, 2021, and titled DEVICES, METHODS AND SYSTEMS TO COLLECT, STORE, AND ANALYZE CHEMICAL SUBSTANCES, which is hereby incorporated by reference in its entirety for all purposes; PCT Patent Application Publication WO 2022/133276 A1, filed on Dec. 17, 2021, and titled DEVICES, METHODS AND SYSTEMS TO COLLECT, CONCENTRATE, STORE, AND ANALYZE CHEMICAL SUBSTANCES, which is hereby incorporated by reference in its entirety for all purposes.

The cartridge may be placed into the device for analyzing the reactant array (e.g., via an opening in the device) during and/or through pre and post exposure to fluid for optimal reactant array image/reflectometer analysis. That is, analysis of the reactant array in the cartridge may include analyzing the reactant array prior to exposure to the fluid, during exposure to the fluid, and/or after exposure to the fluid is complete. Once the analysis of the reactant array in the cartridge is complete, the cartridge and tubing (or specimen or carrier) coupled to or thereof may be removed from the device for analyzing the reactant array and discarded or cleaned for reuse (e.g., the reactant array may be disposed of and the cartridge may be cleaned). Having the cartridge and tubing be disposable may prevent cross-contaminations of the CSA and erroneous analyses (e.g., due to the previous fluids persisting in the tubing and reflowing over the CSA, undesirably flowing onto a human, etc.).

To prevent contamination due to reuse of a cartridge, the cartridge may include a single-use feature that indicates whether the cartridge has been previously used for a fluid analysis. In some configurations, the single-use feature, when included, may be or may include at least one visual indicator or other indicator that indicates the cartridge has been used for a fluid analysis, where a device configured to analyze a reactant array of the cartridge may prevent use of the cartridge if it determines from the indicator that the cartridge has been used.

The analysis system may be an open-loop system or a closed-loop system. In open-loop systems, a fluid flow (e.g., a flow of nitrogen, a gas at a known relative humidity, ambient air, and/or other suitable gasses) may be passed over a target area (e.g., an infection and/or other suitable target area) which then transports analytes (e.g., as produced by bacteria and/or otherwise emitted) at or from the target area to the cartridge and over and/or through the reactant array such that reactants of the reactant array react with the analytes, and outputs the analytes to waste (e.g., to atmosphere, to a waste collection component, and/or other suitable component). An open-loop system as described or otherwise, may be inefficient due to not all of the analytes present in the flowing fluid interacting with the reactants of the reactant array, as the analytes only make a single pass over or through the reactant array.

To maximize the interaction of analytes with the reactants of the reactant array and minimize expelling analytes into an ambient environment and/or a waste container, the output or outlet of the cartridge may be connected back to the target site and/or an input of the cartridge to increase a concentration of analytes passing over or through the reactant array. Although other configurations are contemplated, an example closed-loop cartridge or fluid sensing system is described in PCT Published Appl. No. WO 2022/099021 A1, which has been incorporated by reference herein in its entirety for all purposes.

To facilitate moving the fluid from a collector, specimen, carrier, or target area (e.g., from a sample source) and/or for other suitable purposes, an inline pump and/or tank or cylinder of gas (e.g., N2 and/or other suitable gas) may be utilized to push and/or pull fluid from a target area or collector through the tubes and/or cartridge for passing the fluids over and/or through the CSA. As contamination could occur due to use of a pump contacting fluid internal to the cartridge and/or tubing and reusable tubing within the device for analyzing the CSA and/or other reusable tubing, a reusable pump may be configured to be isolated from the fluid to reduce contamination risks.

A pump or pumping components of a pump that are isolated from the fluid in the tubing and/or the cartridge may be configured in any suitable manner. In one example configuration, at least a portion of the pump may be part of the device for analyzing the reactant array and configured to interact with a pliable portion of the tubing and/or the cartridge received in the device. For example, a portion of a pump may act (e.g., with a peristaltic or other suitable motion) against a pliable portion (e.g., a diaphragm) of the tubing and/or cartridge received at the device to pump fluid through the cartridge. In another example, a portion of the pump may be integrated into or part of the cartridge (e.g., the pliable diaphragm or portion and/or other portion of a pump) and a portion of the pump may be integrated into or part of the device for analyzing the reactant array. For example, an impeller, propeller, Archimedes screw, piezoelectric mechanism, plunger, diaphragm and/or other suitable driven component may be integrated into the cartridge and configured to engage with a drive component of the device for analyzing the reactant array. In some cases, an axle or rod defining or in communication with the driven component of the cartridge may protrude from the cartridge, while keeping a hermetic seal around the fluid in the cartridge, and engage a motor (e.g., a drive component) in the device for analyzing the reactant array that is configured to move (e.g., rotationally, axially, etc.) the axle or rod and accordingly, drive (e.g., drive rotationally, axially, etc.) the driven component of the cartridge. Additionally or alternatively, the cartridge may include mechanical (e.g., flexible diaphragms manually or automatically actuated, plugs, plug receptacle, etc.), electrical (e.g., metallic or other suitable electrically conductive components), optical, and/or magnetic contacts that engages an associated mechanical (e.g., a flexible membrane, a rigid actuator, etc.), electrical, optical, and/or magnetic contact on the device for analyzing the reactant array such that the driven component of the pump (e.g., a piezoelectric component, a flexible diaphragm, a fluid in a flexible reservoir, etc.) is actuated in response to engagement of the respective contacts and/or signals passing between the contacts. Such mechanical, electrical, optical, and/or magnetic contacts may allow for avoiding a leak point at an opening of the cartridge through which an axle, for example, may extend.

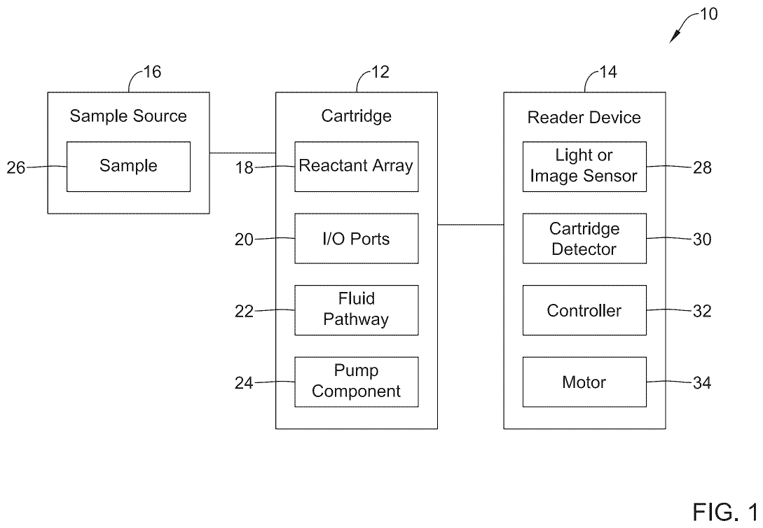

Turning to the Figures, FIG. 1 depicts a schematic diagram of an illustrative system 10 for analyzing a reactant array. Among other components the system 10 may include a cartridge 12 having the reactant array 18 and a reader device 14 configured to monitor and/or analyze the reactant array 18 (e.g., a device for analyzing a CSA or reactant array 18). In some configurations, the system 10 may include a sample source 16 configured to collect, obtain, and/or provide a sample (e.g., a solid, a fluid, etc.) from a target area (e.g., a wound, pollen from a flower, an infection, an exhalation from a subject, a sweat gland, etc.) for analysis using the cartridge 12 and/or the reader device 14. Although illustrative sizes, shapes, and configurations of the cartridge 12, the reader device 14, the sample source 16, and components thereof are discussed herein and depicted herewith, the cartridge 12, the reader device 14, the sample source 16, and the components thereof may have any suitable shape, size, and/or configuration.

The cartridge 12 may have any suitable configuration and may include any suitable components configured to facilitate receiving fluid from the sample source 16, sensing analyte of the fluid from the sample source 16, and interfacing with the reader device 14. Example components of the cartridge 12 include, but are not limited to, one or more reactant arrays 18, one or more input/output (I/O) ports 20, one or more fluid pathways 22, one or more pump components 24, and/or one or more other suitable components and/or configurations. Components that the cartridge 12 may include but may not be depicted in FIG. 1 include, but are not limited to, a housing, a window for viewing the reactant array 18, one or more compartments, one or more gaskets or other features for hermetically sealing the one or more compartments, one or more access openings extending between the compartments and exterior of the housing, one or more seals, valves, caps (e.g., snap caps, screw caps, plug caps, etc.), foil, pierceable membranes, etc. configured to seal the I/O port(s) 20, one or more doors or lids, one or more motors for pumping fluid to the reactant array 18, tubing, one or more filters, one or more diaphragms or membranes, one or more single-use components, and/or other suitable components.

Although not depicted in FIG. 1, the cartridge 12 may include the housing, where the housing may be configured to entirely or at least partially enclose or house the reactant arrays 18 and the fluid pathway 22. The housing may be a single component with one or more openings between the fluid pathway 22 and an exterior of the cartridge 12 and/or a plurality of components defining the opening(s) between the fluid pathway 22 and the exterior of the cartridge 12.

The housing may be formed in any suitable manner. In some examples, the housing may be formed with one or more molding techniques, injection molding techniques, welding techniques, ultrasonic welding techniques, three-dimensional (3D) printing techniques, and/or other suitable techniques.

The housing of the cartridge 12 may be formed from any suitable material. Example suitable materials include, but is not limited to, polymers, metals, glass, and/or other suitable types of materials.

The housing of the cartridge 12 may have any suitable shape and/or size configured to receive fluid and/or a sample source and facilitate analyzing the reactant array in the cartridge 12 using the reader device 14. For example, the cartridge 12 may have a cube shape, an elongated shape, a rectangular shape, an oval shape, a rounded shape, a circular shape, a ball shape, and/or other suitable shape.

The reactant array 18 may be any suitable array of one or more reactants (e.g., analyte sensitive material) and the reactants of the reactant array 18 may be formed from any suitable material. In some cases, the reactant array 18 may be part of or form a colorimetric sensor array (CSA). The cartridge 12 (e.g., the housing of the cartridge 12) may be configured to entirely or at least partially house or enclose the reactant array 18 (e.g., house the CSA including the reactant array 18).

The material of the reactants of the reactant array 18 may be reversible (e.g., reusable), semi-reversible, or non-reversible (e.g., single use). In some examples, the material of the reactants may be an optically responsive chemical material (e.g., a chemoresponsive material) that changes color in response to detecting one or more analytes (e.g., volatile compounds, gases, liquids, and/or other fluids) in a fluid to which the reactants are exposed, but other suitable material is contemplated. Example suitable materials for reactants include dyes from, but not limited to, the following classes: Lewis acid/base dyes (e.g., metal containing dyes), Brensted acidic or basic dyes (e.g., pH indicators), dyes with large permanent dipoles (e.g., solvatochromic dyes), redox responsive dyes (e.g., metal nanoparticle precursors), and/or other suitable classes of dyes. One example material for the reactants may be a silver nanoparticle material. Other suitable materials for the reactants are contemplated, including reactant material other than a printed dye or an optically responsive chemical material.

The reactants of the reactant array 18 may be applied to a substrate. When the reactant array 18 forms or is otherwise part of a CSA, the substrate may or may not be part of the CSA. Additionally or alternatively, the substrate may be the cartridge 12 (e.g., the housing of the cartridge and/or other suitable component of the cartridge) or a component configured to be received in the cartridge 12 (e.g., in one or more of compartments of the cartridge 12).

The reactants of the reactant array 18 may be applied to a substrate in any suitable manner. In one example, the reactants may be applied to the substrate by printing the reactants (e.g., the material of the reactants) on the substrate. When printed, any suitable printing techniques may be utilized including, but not limited to, pin transfer, inkjet, silkscreen, and/or other suitable application techniques.

The reactants may be applied to the substrate randomly and/or to form one or more patterns. Example configurations of the reactants of the reactant array applied to the substrate include, but are not limited to, grid patterns of rows and columns, concentric rings, color matching of a color of printed dye material with a color of a substrate material prior to interactions with analyte, patterns that result in identifiable shapes when the analyte sensitive material reacts to a particular analyte, other suitable configurations, and/or combinations thereof.

The one or more I/O ports 20 may be any suitable types of ports, where the input ports and the output ports may be similar to or different than one another. The one or more input ports may be any suitable port configured to receive fluid from the sample source 16 and facilitate fluidly coupling the sample source 16 with the fluid pathway 22. The one or more output ports may be any suitable port configured to output fluid from the fluid pathway 22 and facilitate fluidly coupling the fluid pathway 22 with a waste location (e.g., the ambient, a waste container, etc.) and/or the sample source 16. In some configurations, the I/O port(s) 20 may be configured to couple with tubes of or in fluid communication with at least a portion of the sample source 16, one or more carriers including a sample, one or more specimens with a sample thereon, and/or other suitable sample sources 16.

The I/O port(s) 20 may be configured to couple with a component of the sample source 16 or a component in communication with the sample source 16 or a waste location in any suitable manner. For example, the I/O port(s) 20 may be configured to couple with one or more components using a friction fit, a luer lock, a ball-detent, threads, a bayonet lock, an O-ring, and/or other suitable coupling techniques or configurations. In some examples, the coupling between the I/O port(s) 20 and components of or in communication with the sample source 16 or waste location may be a fluid tight coupling, but other suitable configurations are contemplated. In one example, a connector of a tubing set may couple with the I/O port(s) 20 using a fluid tight friction fit connection. In another example, a connector of a tubing set may couple with the I/O port(s) 20 using a fluid tight luer lock connection.

One or more seals and/or valves may be located at or in the I/O ports 20. The seals and/or valves may be configured to fluidly seal (e.g., hermetically seal) the fluid pathway 22 from an ambient environment prior to, while, and/or after the sample source 16 engages the I/O port(s) 20.

The one or more seals and/or valves may be or include a single valve (e.g., a check valve and/or other suitable type of valve) or multiple valves, which may be located across a fluid path of the I/O port(s) 20. The one or more valves may include a slit or other access location biased closed and that may be opened in response to engagement with the sample source 16. When the sample source 16 or a component in communication with the sample source 16 extends through the valve, the valve may be configured to seal around the sample source 16, but this is not required.

Alternatively or additionally to utilizing valves, one or more seals (e.g., a single-use seal, such as a foil seal, a burstable membrane, and/or other suitable seals) may be utilized at or proximate to the I/O port(s) 20 to seal the fluid pathway 22 from the ambient environment. In some examples, a single-use seal may extend across an opening or pathway through the I/O port(s) 20 and may be pierced or broken in response to coupling tubing or other components with the I/O port(s) 20. In some examples, the single-use seal may be a peel-away seal configured to be peeled off of the I/O port(s) 20 prior to coupling tubing and/or other components with the I/O ports 20.

The one or more fluid pathways 22 of the cartridge 12 may extend from the input port to the output port of the I/O ports 20 and may or may not include a fluid path through the I/O ports 20. The fluid pathway 22 may include a tubing portion, a compartment portion, a pump portion, combinations of portions (e.g., tubing portion and a pump portion may be a same portion of the fluid pathway 22) and/or other suitable portions. In some examples, the fluid pathway 22 may include a tube portion, a pump portion, and a compartment portion, where the tube portion may include one or more tubes or compartments defining the fluid pathway 22 from the input port to a pump chamber of or at the pump portion extending from the tube portion, through the pump chamber, and to the cartridge portion extending to the output port. The cartridge portion or other suitable portion of the fluid pathway 22 may include the reactant array 18. In some examples, the fluid pathway 22 may include a tube portion and a compartment portion, where the tube portion may include one or more tubes or compartments defining the fluid pathway 22 from the input port to the compartment portion extending from the tube portion to the output port such that a pump may engage the tube portion to pump fluid from the input port through the tube portion, over or to a reactant array 18 in the compartment portion, and out of the output port. In some examples, the fluid pathway 22 may include a tube portion extending from the input port to the output port, where the tube portion may include one or more tubes defining the fluid pathway 22 and a pump may engage the tube portion to pump fluid from the input port through the tube portion to a reactant array in the tube portion.

Although the fluid pathway 22 is depicted in the Figures as passing a pump component and the passing the reactant array 18, it is contemplated that the fluid pathway 22 may pass the reactant array 18 prior to passing the pump component. Additionally or alternatively, the fluid pathway 22 may fluid over the reactant array 18 from one or more directions, as desired. In some configurations, the fluid pathway 22 may be separated to separately flow over and/or through individual reactants of the reactant array 18.

The tubing of the tubing portion of the fluid pathway 22 may be any suitable type of tubing and/or inflow compartment of the cartridge 12. The tubing of the tubing portion of the fluid pathway 22, when include, may be flexible, resilient, pliable, rigid, and/or have one or more other suitable properties. In some examples, at least part of the tubing portion may be flexible, pliable, and/or resilient material such that the flexible, pliable, and/or resilient material may be a diaphragm or membrane that interacts with a pump component to facilitate pumping fluid through the fluid pathway 22. Alternatively or additionally, at least part of the tubing portion may be configured from channels in the housing of the cartridge 12. Other suitable configurations are contemplated.

The pumping portion of the fluid pathway 22 may be formed in any suitable manner. In some examples, the pumping portion of the fluid pathway 22 may include a pumping chamber, a diaphragm, a piston, and/or other suitable pumping components. In one example, the pump portion of the fluid pathway 22 may include a pumping chamber receiving fluid from the tubing portion and providing fluid to the compartment portion, where a pump component fluidly isolated from the fluid pathway 22 may act an on the diaphragm to draw fluid into the pumping chamber and/or to output fluid from the pumping chamber. The pumping chamber, when included, may be at least partially defined by the housing of the cartridge 12, a diaphragm, a piston, and/or other suitable components.

The compartment portion of the fluid pathway 22 may comprise one or more compartments and may be entirely or at least partially defined by the housing of the cartridge 12 and/or other suitable components. The one or more compartments may be sized or otherwise configured to include or receive the reactant array 18, fluid from an upstream portion of the fluid pathway 22 (e.g., the input port, tube portion, pump portion, etc.) and output fluid through the output port(s).

Although not depicted in FIG. 1, the cartridge 12 may include one or more filters in the fluid pathway 22 and/or the I/O ports 20. The one or more filters may be located in the tubing portion, the pump portion, the compartment portion, and/or other portions of the fluid pathway 22. In some examples, the one or more filters may be configured to remove undesirable molecules from the fluid passing through the fluid pathway 22 and allow desirable molecules to pass through the filter(s). In some examples, the one or more filters may be configured to interact with particular molecules to enhance reactivity of the reactant array 18 with the fluid passing through the pathway and/or to alter incoming fluid and/or analytes thereof into different constituents for greater sensitivity at the reactant array 18. The filter may be sized, shaped, doped or impregnated with different constituents, and/or otherwise configured differently to address filtering out different molecules. Some variables that may be adjusted to configure the filter include, but are not limited to, material of the filter, pore sizes in the filter, pore concentrations in the filter, pore locations in the filter, etc.

One or more valves may be located in the fluid pathway 22. In some examples, a valve or valve component may extend between each portion of the fluid pathway 22, but other suitable configurations are contemplated. Example suitable valves include, but are not limited to, check valves, one-way valves, rubber flaps, plastic flaps, ball/spring valves, umbrella valves, and/or other suitable types of valves.

The cartridge may include a pump or at least one or more pumping components 24. In some cases, the pump or at least one or more pumping components 24 of the cartridge 12 may be fluidly isolated from the fluid traversing the fluid pathway 22. Alternatively or additionally, the pump or at least one or more pumping components 24 may be in fluid communication with the fluid traversing the fluid pathway 22.

The pumping components 24 may be any suitable components configured to facilitate moving fluid along the fluid pathway 22. Example suitable pump components 24 include, but are not limited to, a flexible diaphragm or portion of tubing defining the fluid pathway 22, a flexible diaphragm, an impeller, a bellows, a propeller, an Archimedes screw, a piezoelectric mechanism, a plunger, a piston, an axel or extension, a coupler for coupling with other pump components, and/or other suitable pump components. In some configurations, the pump component 24 may be a driven component configured to be driven by a drive component of the reader device 14. For example, a driven component (e.g., an impeller, propeller, Archimedes screw, piezoelectric mechanism, plunger, and/or other suitable driven component) may be integrated into the cartridge 12 and configured to engage with a drive component of the reader device 14 (e.g., a motor, an actuator, a drive shaft, etc.) In some examples, an axle defining or in communication with the driven component of the cartridge 12 may protrude from the cartridge 12, while keeping a hermetic seal around the fluid in the fluid pathway 22, and engage a motor or drive shaft of the motor (e.g., a drive component) in the reader device 14 that may be configured to drive (e.g., rotate) the axle and move the driven component of the cartridge 12. Additionally or alternatively, the cartridge 12 may include a mechanical (e.g., flexible diaphragms manually or automatically actuated, etc.), electrical (e.g., metallic or other suitable electrically conductive components), and/or magnetic contacts that engages (e.g., directly or indirectly) an associated mechanical, electrical, and/or magnetic contact on the reader device 14 such that the driven component of the cartridge 12 may be actuated in response to engagement of the respective contacts and/or signals passing between the contacts. Such mechanical, electrical, and/or magnetic contacts may allow for avoiding a leak point at an opening of the cartridge through which an axle, for example, may extend.

The cartridge 12 may include a single-use component configured to prevent contamination due to reuse of a cartridge. In some cases, the single-use component may be a mechanical, electrical, electromechanical, optical, chemical, magnetic, and/or other suitable type of single-use feature that changes in response to being used with a single-use or reusable reader device 14 for analyzing the reactant array 18. In one example of a single-use component for a cartridge 12, an electronic single-use component may be imbedded within the cartridge 12 and either recognized by the reader device 14 as being intact or broken to indicate whether the cartridge 12 is new or has been used, respectively. Example electronic single-use components include, but are not limited to a memory devices, radio-frequency identification (RFID) devices, a fuses, and/or other suitable electronic single-use components. When a memory device is used as a single-use component of the cartridge 12, the memory device may be accessed by the reader device 14 and modified by the reader device 14 after the cartridge 12 has been inserted into the reader device 14. When a fuse is used as a single-use component, the fuse may be broken (e.g., via excessive current or in other suitable manners) after the reader device 14 analyzes the reactant array 18, where the reader device 14 may be configured to identify the broken fuse and the cartridge 12 with the broken fuse may be prevented from being used in a subsequent analysis. Further, the single-use component may be or may include at least one visual indicator that may change state after use in the reader device 14 and that may be captured by an image capture sequence of the reader device 14, where the reader device 14 may prevent use of the cartridge 12 if it determines from the indicator that the cartridge 12 has been used.

In some cases, the cartridge 12 may be reusable. For example, in instances when the reactant array 18 and/or other components of the cartridge 12 that may be spent or contaminated with a received fluid (e.g., a filter, etc.), the reactant array 18 and/or other spent or contaminated components of the cartridge 12 may be removed from the cartridge 12 and the remaining components of the cartridge 12 may be reused after cleaning, as needed. In some cases, the reactant array 18 include reversible reactant material and the reactant array 18 may be reused with other components of the cartridge 12.

Although the cartridge 12 may include a single fluid pathway 22 extending between an input port and an output port and to, through, and/or over the reactant array, the cartridge 12 may include a plurality of fluid pathways 22 that are each configured to bring fluid to a same or different reactant array 18. In one example, the cartridge 12 may include a first input port, a first output port, a first fluid pathway 22 extending between the first input port and the first output port, and a first reactant array 18 in the first fluid pathway 22, along with up to an nth (e.g., an integer number greater than one and all integers between one and the nth number) input port, an nth output port, an nth fluid pathway 22 extending between the nth input port and the nth output port, and an nth reactant array 18 in the nth fluid pathway 22. Further, in some configurations, one or more pump components 24 may be configured to interact with the first through nth fluid pathways 22, such that multiple fluid pathways 22 may share a pump component 24 and/or one or more of the first through nth fluid pathways 22 have a pump component 24 dedicated to that fluid pathway 22 only. When the cartridge 12 is so configured, the cartridge 12 may be configured to be used to analyze multiple different fluids (e.g., fluids from different or same samples) in parallel or simultaneously using one or more reader devices 14.

The sample source 16 may be configured to collect, collect, and/or provide a sample 26 from a target area or fluid from the sample 26 or target area to the cartridge 12, where the sample 26 or target area may be or may be configured to exude analytes. In some examples, the sample source 16 may be a carrier (e.g., a container, a compartment, etc.) configured to receive the sample 26, a specimen (e.g., swabs, swabs on a stick, sponges, sample plates, test strips, etc.) configured to collect a sample from a target area, a collector configured to collect fluid (e.g., fluids containing analytes) from a target area, a concentrator, and/or other suitable components configured to provide sample or fluid from a sample to the cartridge. In some examples, the sample source 16 may include or may be coupled with tubing configured to transport fluid from the sample source 16 or target area to the cartridge 12, but other suitable configurations are contemplated.

The reader device 14 may include one more suitable components for reading and/or analyzing the reactant array 18, which may be in or separate from the cartridge 12. Example suitable components of the reader device 14 include, but are not limited to, illumination components, light collection components, one or more light or image sensor 28, one or more cartridge detector 30, one or more controllers 32, one or more motors 34, one or more light sources, one or more sets of lenses, one or more pumps, one or more buttons, one or more user interfaces, one or more displays, and/or other suitable components. The controller(s) 32, the motor(s) 34, and/or other components of the reader device 14 may be an actuator for the pump component 24 of the cartridge 12. In some examples, the reader device 14 may be a bench top device or a handheld device, each configured to be used with the cartridges 12 discussed herein or otherwise and/or to analyze the reactant arrays 18 thereof. In some cases, the reader device 14 may be isolated from the sample 26 and/or fluid from the sample 26 or target area and may be configured for reuse.

The reader device 14 may be powered with any suitable power source. Example suitable power sources for powering the reader device 14 include, but are not limited to, battery power in the reader device 14, solar power at the reader device 14, wall or line power, and/or other suitable power sources. In some examples, to facilitate forming the reader device 14 with a handheld configuration, the reader device 14 may be powered by one or more batteries and/or by solar power.

The light or image sensor 28 may be and/or may include one or more light collectors of any suitable type. Example suitable types of light collectors may include, but are not limited to, a light sensor, an image sensor, an n-dimensional sensory array (e.g., where “n” equals 1, 2, etc.), a linear 2D light detector array image sensor, light detector array image sensor may, a spectrometer, a refractometer, a charge-coupled device (CCD) image sensor, complementary metal-oxide semiconductor (CMOS) image sensor, contact image sensor (CIS), color contact image sensor (CCIS), a camera, other suitable light collectors, and/or combinations of light collectors. In one example, the light collector may include or may be a spectrometer configured to measure photons collected from (e.g., reflected, transmitted, and/or otherwise received from) the target area. Utilizing a spectrometer may facilitate sensing wavelengths of light with high resolution in the nanometer range and may provide a continuous set of data over the wavelength range, which allows for a sensitive analysis of the data to identify components of a fluid to which the reactant array 18 was exposed relative to when other light collectors are used. In another example, the light collector may include a 2D pixel array image sensor configured to record multiple spatial interferograms in a pixel array direction of an interferogram representing a Fourier transform of the reactant array 18, which may provide sufficient sensitivity, while being compact and cost-effective.

The reader device 14 may include one or more cartridge detectors 30. In some examples, the cartridge detector 30 may be configured to mechanically, electrically, optically, magnetically and/or use combinations thereof (e.g., electromechanically, opto-electronically, etc.) or other suitable techniques to detect when the cartridge 12 is proximate and/or received in the reader device 14. In one example, the cartridge detector 30 may be a pin or button or other component, where the pin or button or other component physically or mechanically engages the cartridge 12 as the cartridge 12 is inserted into or positioned at the reader device 14 and in response, the pin or button or other component adjusts and provides a mechanical and/or electrical (e.g., completes a circuit, etc.) indication that the cartridge 12 has been received. Alternatively or additionally, the cartridge detector 30 may be an electronic sensor or detector and/or other suitable type of detector that may provide an indication to the reader device 14 indicating that cartridge 12 is proximate and/or has or has not been received in or at the reader device 14 (e.g., has or has not been properly received in the cartridge 12). In some cases, the electronic sensor or detector may sense a signal from the cartridge 12 (e.g., an RF signal), sense the cartridge 12 breaking a circuit of the reader device 14 or the cartridge 12, completing a circuit of the reader device 14 and/or the cartridge 12, and/or sense a presence of the cartridge 12 in one or more other suitable manners.

In addition to or as an alternative to the cartridge detector 30, the reader device 14 may include a single-use component. The single-use component of the reader device 14 may include a feature configured to electrically, mechanically, or electrically and mechanically modify the cartridge 12 such that the cartridge 12 will not be used more than once by the reader device 14 (e.g., in more than one test by the reader device 14). Example single-use components include, but are not limited to, a component configured to write to an RFID tag of the cartridge 12, a component configured to mechanically alter the cartridge 12 to prevent recoupling of the cartridge 12 after the cartridge 12 has been removed from the reader device 14, a component configured to optically alter the cartridge after insertion into the reader device 14, a camera configured to read a code (e.g., a bar code, QR code, alphanumerical code, color code, etc.) on a surface of the cartridge 12 or an RFID reader configured to read a code from an RFID of the cartridge and add the code or other identifying material to a list of used cartridges in memory of the reader device 14 and/or in communication with the reader device 14, a component configured to break a fuse of the cartridge 12, and/or other components configured to mark the cartridge 12 as used.

The cartridge detector 30 and/or other suitable detector of the reader device 14 may be configured to detect when the sample source 16 and/or the sample 26 is fluidly coupled with the cartridge 12. For example, the cartridge 12 may be inserted into the reader device 14 before the sample source 16 is fluid coupled with the cartridge 12 and the cartridge detector 30 may detect when the sample source 16 is becomes fluidly coupled with the cartridge 12 after the cartridge 12 is inserted into the reader device 14. Upon sensing the cartridge 12 in the reader device 14 and the sample source 16 fluidly coupled with the cartridge 12, the reader device 14 may automatically initiate a light or image collection from the reactant array 18 as part of a fluid analysis test of fluid from a sample at the sample source 16 and/or automatically request input from a user to perform one or more steps of the fluid analysis test.

The motor(s) 34 of the reader device 14 may be any suitable type of motor. Example suitable types of motors 34 include, but are not limited to, stepper motors, electric motors, brushed motors, brushless motors, induction motors, magnetic motors, servo motors, and/or other suitable types of motors. The motor(s) 34 of the reader device 14 may include a drive shaft and/or other suitable drive component configured to engage a driven component of the cartridge to pump fluid through the fluid pathway 22. Alternatively or additionally, the motor, the drive shaft, and/or other suitable drive component of or in communication with the motor(s) 34 may be a pump component configured to engage or be in communication with a pump component (e.g., a flexible diaphragm, motor coupler, etc.) of the cartridge 12 (e.g., defining the fluid pathway 22). In some examples, the motor, the drive shaft, and/or other drive component of the reader device 14 may include a coupling component (e.g., electrical, mechanical, and/or magnetic coupling components) configured to couple with or otherwise mate with a pump component of the cartridge 12.

The controller 32 of the reader device 14 may be configured to control operations of the reader device 14 in response to receiving one or more control signals and/or use inputs. The controller 32 may store captured data at the reader device 14 and/or send data to a remote storage component for storage and the controller 32 may use stored captured data to analyze the reactant array 18. Further the controller 32, may be implemented entirely on the reader device 14, partially on the reader device 14 and partially remotely, and/or entirely remotely (e.g., on a server or other suitable computing device, on the cartridge 12, on a user's mobile device, etc.)

The controller 32 of the reader device 14 may be coupled to one or more other electronic components of the system 10. For example, the controller 32 may be communicatively coupled with one or more of the illumination components, when included, the light or image sensor 28, the cartridge detector 30, the motor 34, the single-use component, and/or one or more other suitable components of the system 10 and/or remote components (e.g., servers, mobile devices, etc.) that may or may not be part of the system 10. In some examples, the controller 32 may be configured to receive an indication to initiate a fluid test (e.g., from a user via a user interface of or in communication with the controller 32, from the cartridge detector 30, etc.) and send coordinated control signals to one or more electronic components of the system 10.

The controller 32 may be configured to identify or may facilitate identifying a component of fluid in contact with the reactant array 18 and/or a condition of a target area based on measured (e.g., sensed and/or calculated) levels of light (e.g., interferograms, images, reflectance, etc.) or changes in light sensed or collected from the reactant array 18 with the light or image sensor 28. In some examples, the controller 32 may be configured to identify a component of fluid in contact with the reactant array 18 and/or a condition of the target area based on one or more of a timing of levels of light from the reactant array 18 and an absolute change between a level of light from the reactant array 18 at a time of or prior to coupling the sample source 16 with the cartridge 12 and at a predetermined or an indeterminate time (e.g., based on reactant array responses, analyte concentrations, fluid flow rate, fluid temperature, etc.) after coupling the sample source 16 with the cartridge 12, and levels of light from the reactant array 18 relative to predetermined or expected levels of light from the reactant array 18. The controller 32 may be configured to identify the component of the fluid in contact with the reactant array 18 and thus, a component of the sample 26 and/or a condition at a location from which the sample was taken (e.g., a wound, pollen from a flower, an infection, an exhalation from a subject, a sweat gland, etc.) based on light from the reactant array 18 that is received at the light or image sensor 28 in one or more additional or alternative manners.

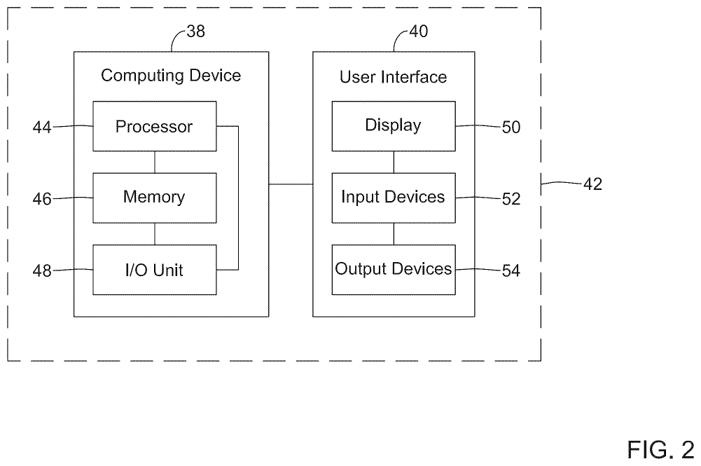

The controller 32 and/or other components of the system 10 may be or may include one or more computing devices including or coupled with one or more user interfaces. FIG. 2 depicts a schematic diagram of an illustrative computing device 38 and a user interface 40, where the computing device 38 and/or the user interface 40 may be entirely or partially housed in one or more housings 42 (e.g., a housing which may or may not house other components of the system 10). The housing 42 may be an optional component, as represented by the broken lines defining the housing 42 depicted in FIG. 2. Although various components are depicted as being included in the computing device 38 and the user interface 40, one more of the depicted components may be omitted and/or one or more additional or alternative components may be utilized.

The computing device 38 may be any suitable computing device configured to process data of or for the system 10 and may be configured to facilitate operation of the system 10. The computing device 38, in some cases, may be configured to control operation of the system 10 by establishing and/or outputting control signals to the light or image sensor 28 and/or other electronic components of the system 10 to run a test on fluid from the sample 26 at the sample source 16 that interacts with the reactant array 18 and/or monitor results of a test. In some examples, the computing device 38 may be part of the controller 32 and may communicate with other components over a wired or wireless connection, but other suitable configurations are contemplated. When the computing device 38, or at least a part of the computing device 38, is a component separate from a structure of the controller 32, the computing device 38 may communicate with electronic components of the system 10 over one or more wired or wireless connections or networks (e.g., LANs and/or WANs). In some cases, the computing device 38 may communicate with a remote server or other suitable computing device.

The illustrative computing device 38 may include, among other suitable components, one or more processors 44, memory 46, and/or one or more input/output (I/O) units 48. Example other suitable components of the computing device 38 that are not specifically depicted in FIG. 2 may include, but are not limited to, communication components, a touch screen, selectable buttons, and/or other suitable components of a computing device. As discussed, one or more components of the computing device 38 may be separate from the controller 32 and/or incorporated into the components of the controller 32.

The processor 44 of the computing device 38 may include a single processor or more than one processor working individually or with one another. The processor 44 may be configured to receive and execute instructions, including instructions that may be loaded into the memory 46 and/or other suitable memory. Example components of the processor 44 may include, but are not limited to, central processing units, microprocessors, microcontrollers, multi-core processors, graphical processing units, digital signal processors, application specific integrated circuits (ASICs), artificial intelligence accelerators, field programmable gate arrays (FPGAs), discrete circuitry, and/or other suitable types of data processing devices.

The memory 46 of the computing device 38 may include a single memory component or more than one memory component each working individually or with one another. Example types of memory 46 may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory 46 may be or may include a transitory and/or non-transitory computer readable medium. The memory 46 may include instructions stored in a transitory state and/or a non-transitory state on a computer readable medium that may be executable by the processor 44 to cause the processor 44 to perform one or more of the methods and/or techniques described herein. Further, in some cases, the memory 46 and/or other suitable memory may store data received from the light or image sensor 28 and/or other components of or in communication with the system 10.

The I/O units 48 of the computing device 38 may include a single I/O component or more than one I/O component each working individually or with one another. Example I/O units 48 may be or may include any suitable types of communication hardware and/or software including, but not limited to, communication components or ports configured to communicate with electronic components of the system 10 and/or with other suitable computing devices or systems. Example types of I/O units 48 may include, but are not limited to, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless communication components (e.g., radio frequency (RF) components, Low-Energy BLUETOOTH protocol components, BLUETOOTH protocol components, Near-Field Communication (NFC) protocol components, WI-FI protocol components, optical communication components, ZIGBEE protocol components, and/or other suitable wireless communication components), and/or other suitable I/O units 48.

The user interface 40 may be configured to communicate with the computing device 38 via one or more wired or wireless connections. The user interface 40 may include, among other components, one or more display devices 50, one or more input devices 52, one or more output devices 54, and/or one or more other suitable features. Although not depicted, the user interface 40 may include one or more indicators (e.g., light emitting diodes (LEDs), LED linear arrays, numbers, etc.) In some examples, the user interface 40 may be part of or may include the computing device 38. Alternatively or additionally, the user interface 40 may be part of a mobile device or remote computing system.

The display 50 may be any suitable display. Example suitable displays include, but are not limited to, touch screen displays, non-touch screen displays, liquid crystal display (LCD) screens, LED displays, head mounted displays, virtual reality displays, augmented reality displays, a mobile device display, and/or other suitable display types.

The input device(s) 52 may be and/or may include any suitable components and/or features for receiving user input via the user interface 40. Example input device(s) 52 may include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components) and/or other suitable input devices. In one example, the input devices 52 may include a touch screen that allows for setting set points, initiating a fluid or target area analysis test, adjusting between screens (e.g., a testing screen, a data analysis screen, a results screen, etc.), and/or allows for taking one or more other suitable actions.

The output device(s) 54 may be and/or may include any suitable components and/or features for providing information and/or data to users and/or other computing components. Example output device(s) 54 include, but are not limited to, displays, speakers, vibration systems, tactile feedback systems, optical outputs, and/or other suitable output devices.

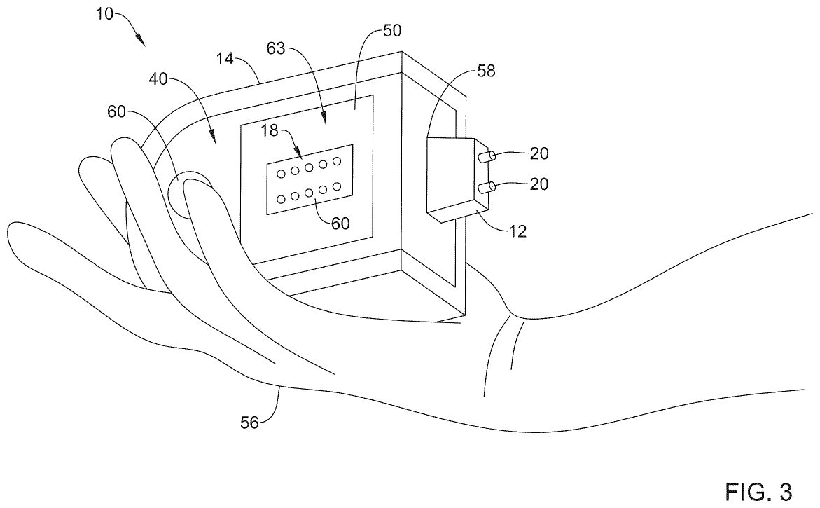

FIG. 3 schematically depicts an illustrative configuration of the system 10 configured to be held in a hand 56 of a user. Although the system 10 may take on a variety of different handheld configurations (and/or non-handheld configurations), the system 10 depicted in FIG. 3 may include the cartridge 12 inserted into the reader device 14 through an access opening 58. The cartridge 12 may include I/O ports 20 for coupling with one or more tubes or components of or in communication with the sample source 16.

The user interface 40 of the reader device 14 depicted in FIG. 3 may include the display 50 and one or more buttons 60. The user interface 40 may include additional and/or alternative components or features including, but not limited to, one or more indicators (e.g., LEDs, LED linear arrays, numbers, etc.) configured to indicate (e.g., as an alert, etc.) a result of a test, a test has been initiated, a test has been completed, a user is to perform an action, an action has been completed, and/or other suitable indications. Additionally or alternatively, the user interface 40 may be on or part of a remote computing device, such as a mobile device, control station, web page, mobile application, and/or other suitable remote computing device. In some examples, the user interface 40 may be entirely omitted from the reader device 14 or one or more components of the user interface 40 discussed herein may be omitted from the reader device 14.

The display 50 (e.g., a touchscreen display or a non-touchscreen display), when included, may depict any suitable images. In one example, the display may depict an image 63 captured by the reader device 14 (e.g., captured by the light or image sensor 28). The image 63 may be a live image and/or a photograph or image captured at a previous time. As schematically depicted in FIG. 3, the image 63 may be a live image of the CSA 66 including the reactant array 18. Further, the display 50 may display material other than the image 63 including, but not limited to, instructions for testing a fluid, a test status (e.g., a progression of steps in an analysis of the reactant array 18), a system status, results of and/or data from an analysis of the reactant array 18, marketing indicia, brand indicia, videos, user pictures, art work, etc.

The one or more buttons 60, when included, may be selected by a user to cause the reader device 14 and/or the cartridge 12 to take one or more actions. For example, a user may interact with the one or more buttons 60 to initiate a pump of the reader device 14 and/or of the cartridge 12, initiate a motor of the reader device 14, initiate an analysis of the reactant array 18, initiate the light or image sensor 28 to take an image or capture light, eject the cartridge 12, mark the cartridge 12 used, initiate the display 50, and/or to cause the reader device 14 and/or the cartridge 12 to take one or more other suitable actions.

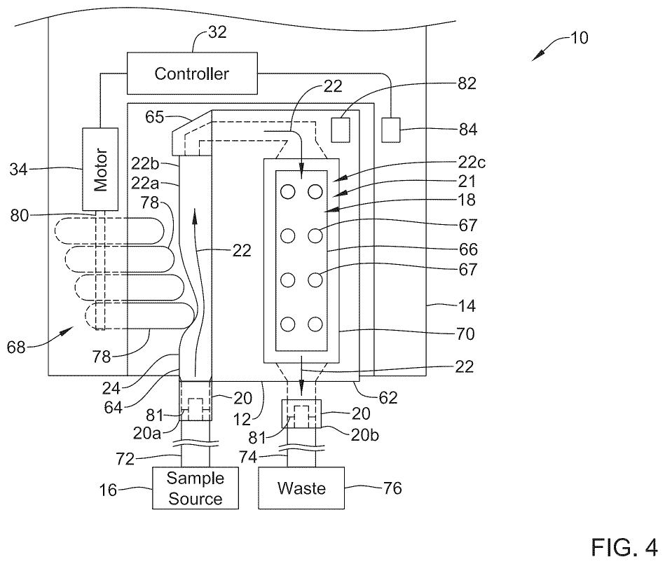

FIGS. 4-12 schematically depict illustrative configurations of cartridges 12, which may be used with handheld reader devices 14 and/or other configurations of the reader device 14. Generally, broken lines in the Figures represent components internal to a housing, unless expressly indicated otherwise.

The illustrative cartridges 12 may include the I/O ports 20 and a housing 62 having a transparent portion 70 (e.g., a window formed from glass, polymer, and/or other suitable transparent material, etc.) through which the reader device 14 may view, collect light, and/or image the reactant array 18 positioned within the cartridge 12. A fluid pathway 22 through the cartridge 12 may extend from an input port through the cartridge 12, through and/or over reactants 67 of the reactant array 18 and out of the output port. Either I/O port 20 may be an input port or an output port depending on which direction fluid is passed through the fluid pathway 22.

The system 10 may include a pump 68 to pump fluid through the fluid pathway 22. The pump 68 may be entirely located at the cartridge 12, entirely located at the reader device 14, partially located at the cartridge 12 and partially located at the reader device 14, and/located at one or more other suitable locations.

FIG. 4 schematically depicts an illustrative configuration of the system 10 with the cartridge 12 inserted into the reader device 14, where a flexible, resilient, or pliable component 64 defining the fluid pathway 22 may be configured as part of a pump 68 (e.g., to engage with peristaltic pump with fingers 78, rollers, and/or other driving components of the pump 68 at the cartridge 12 or the reader device 14). A first I/O port 20a (e.g., an input port) of the cartridge 12 may be coupled to input tubing 72 coupled with or otherwise in fluid communication with the sample source 16. A second I/O port 20b (e.g., an output port) of the cartridge 12 may be coupled to output tubing 74 that is coupled with or is otherwise in fluid communication with a waste container 76, but the output tubing 74 may be output to ambient and/or one or more other suitable locations.

As depicted in FIG. 4, the system 10 may be considered to have an open-loop configuration due to not returning an output from the second I/O port 20b to the sample source 16 and/or the fluid pathway 22. The system 10, however, may be configured in a closed loop manner by returning the output from the second I/O port 20b to the sample source 16, the fluid pathway 22, and/or other suitable location in fluid communication with the fluid pathway 22.

The I/O ports 20 of the cartridge 12 may include one or more seals or valves 81 configured to fluidly seal the fluid pathway 22 from an ambient environment. As the input and output tubes 72, 74 are engaged, the tubes 72, 74 or connectors thereof may open the seals or valves 81 and the seals or valves 81 may seal around the input and output tubing 72, 74. In some examples, one or more of the seals or valves 81 may be peel-away seals that may be removed from the ports 20 prior to connecting the input and output tubing 72, 74 to the ports 20.

The input and output tubes 72, 74 may be permanent parts of the cartridge 12 (e.g., fixedly secured to the housing 62 and/or the I/O ports 20) or removable from the cartridge 12. When the input and output tubes 72, 74 are a permanent part of the cartridge, the only component in or to be in fluid communication with the sample 26 that may need to be connected to the cartridge 12 is the sample source 16. Alternatively, the sample source 16 may be omitted and ends of one or both of the input and output tubes 72, 74 may be placed at a target site to receive fluid therefrom. When the input and/or output tubing 72, 74 is part of the cartridge 12, cross-contamination to/from a patient, device, user, environment, etc. may be mitigated due to utilizing fewer components that could be reused in error as the input and/or output tubing 72, 74 may be discarded with the cartridge 12. Further, utilizing input and/or output tubing 72, 74 that is a permanent part of the cartridge 12 may reduce opportunities for human error due to a reduced number of required connections.

To facilitate interacting with the pump 68 or other suitable pump of the system 10, the flexible, resilient, or pliable component 64 may be exposed from and attached to the housing 62 of the cartridge 12 such that fingers 78 or other suitable components of the pump 68 may engage the pliable, resilient, and/or flexible component 64 (e.g., a flexible diaphragm) to move fluid along the fluid pathway 22. In addition to or as an alternative to being attached to the housing 62, the flexible, resilient, or pliable component 64 may be separate from or integrated into the housing 62 of the cartridge 12. In some examples, the pliable, resilient and/or flexible component 64 configured to engage the fingers 78 and/or other portions of the pump 68 may be one of the pump components 24 of the cartridge 12.

The pliable, resilient, and/or flexible component 64 may be a sheet of material at least partially defining the fluid pathway 22 with a channel or the housing 62 of the cartridge 12. Alternatively or additionally, the pliable, resilient, and/or flexible component 64 may be part of a tube defining the fluid pathway 22. As depicted in FIG. 4, the flexible, resilient, or pliable component 64 may be a tube coupled with the first I/O port 20a and a distal connector 65 of the housing 62, where the tube may be a permanent part of the cartridge 12 or removable from the cartridge 12.

The reader device 14 may have any suitable components configured to drive the pump 68. In some examples, the controller 32 may be in communication with the motor 34 to drive the motor and cause the drive shaft 80 to rotate, which may result in a pump action. In the example depicted in FIG. 4, rotation of the drive shaft 80 may cause the fingers 78 to rotate and sequentially act on the flexible or pliable component 64 to drive fluid through the fluid pathway 22. The pump components of the reader device 14 may be fluidly isolated from the fluid being pumped through the fluid pathway 22 of the cartridge 12. In some cases, the pump 68 may be omitted and a similar or different pump configured to act on the flexible or pliable component 64 of the cartridge 12 may be integrated into the cartridge 12.

The fluid pathway 22 may have any suitable configuration configured to facilitate passing fluid therethrough. As depicted in FIG. 4, a first portion 22a (e.g., a tube portion, inflow portion, and/or other suitable first portion) of the fluid pathway 22 may extend from the first I/O port 20a to a third portion 22c (e.g., a compartment portion, a reactant portion, an outflow portion and/or other suitable portion) and a second portion 22b (e.g., a pump portion, a diaphragm portion, and/or other suitable portion) may overlap with the first portion 22a at the flexible, resilient, or pliable component 64. In some configurations, as the fluid pathway 22 reaches the third portion 22c (e.g., proximate a location of the reactant array 18 in a compartment 21 of the housing 62), the fluid pathway 22 may widen to facilitate dispersing the fluid from sample source 16. Additionally or alternatively, the fluid pathway 22 may include a funnel shape or configuration from the third portion 22c proximate the reactant array 18 and into the second I/O port 20b. Other suitable configurations are contemplated.

In some illustrative configurations, the cartridge 12 may include a single use component 82, as discussed herein or otherwise. In one example, the single use component may be an RFID tag and a single use component 84 of the reader device 14 may receive a signal from the controller 32 or other component to mark the RFID tag of the cartridge 12 with an indication that the cartridge has been used in a fluid analysis test. After the single use component 82 of the cartridge has been marked used, the single use component 84 of the reader device 14 may read or sense that the cartridge 12 has been used and refuse to perform a test using the previously-used cartridge 12, perform the test and make a note that the cartridge 12 was previously used, and/or take one or more other suitable actions or take no action.

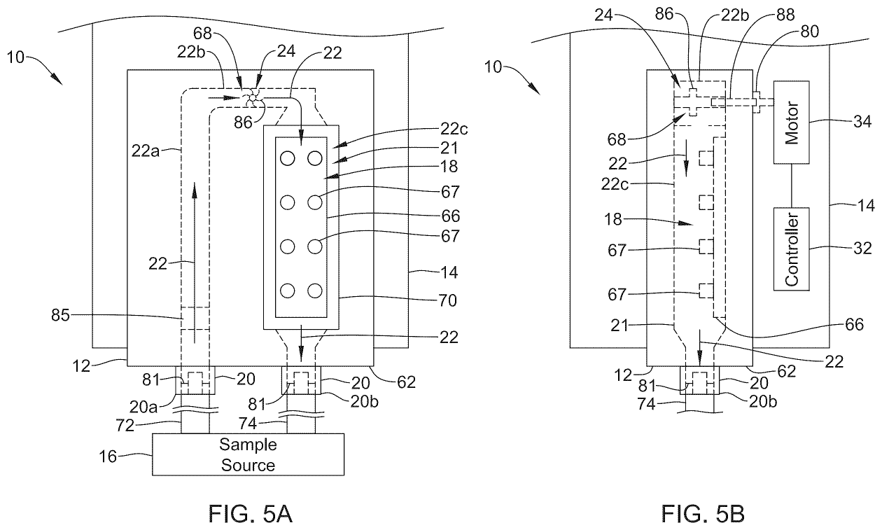

FIGS. 5A and 5B schematically depict top and side views, respectively, of an illustrative configuration of the system 10 with the cartridge 12 inserted into the reader device 14, where a pump component 24 of the cartridge 12 is positioned within the fluid pathway 22. A first I/O port 20a (e.g., an input port) of the cartridge 12 may be coupled to an input tubing 72 coupled with or otherwise in fluid communication with the sample source 16. A second I/O port 20b (e.g., an output port) of the cartridge 12 may be coupled to an output tubing 74 that is coupled with or is otherwise in fluid communication with the sample source 16, but the output tubing 74 may be configured to output fluid to ambient and/or one or more other suitable locations.

As depicted in FIG. 5A, the system 10 may be considered to have a closed-loop configuration due to returning an output from the second I/O port 20b to the sample source 16 and the fluid pathway 22. In some configurations, the system 10 may also be considered a closed-loop configuration when the output from the second I/O port 20b is provided to the target area and the input to the first I/O port 20a receives fluid from the target area.

The cartridge 12 may include a filter 85 in the fluid pathway 22. Although FIG. 5A depicts the filter 85 in the fluid pathway 22 (e.g., a first portion 22a of the fluid pathway 22) proximate the first I/O port 20a, one or more filters 85 may be located at any suitable location along the fluid pathway 22.

The one or more filters 85 in the fluid pathway 22 may be configured to remove undesirable molecules from the fluid passing through the fluid pathway 22 and allow desirable molecules to pass through the filter 85. Additionally or alternatively, the filter 85 may be configured to interact with particular molecules to enhance reactivity of the reactants with the fluid passing through the pathway and/or to alter incoming fluid and/or analytes therein into different constituents for greater sensitivity. The filter 85 may be sized, shaped, and/or configured differently to address filtering out different molecules and/or enhancing detection. Some variables that may be adjusted to configure the filter include, but are not limited to, material of the filter, pore sizes in the filter, pore concentrations in the filter, pore locations in the filter, etc.

As depicted in FIGS. 5A and 5B, the pump 68 may include the motor 34 in the reader device 14 and the pump component 24 in the cartridge 12, among other components, where the pump component 24 may be comprised of a rotating component 86 in the fluid pathway 22 (e.g., at the second portion 22b of the fluid pathway 22) at a location proximal of the third portion 22c of the fluid pathway 22. The rotating component 86 may be or may include an impeller or propeller, but other suitable configurations are contemplated. As the rotating component 86 rotates, fluid may be advanced through the fluid pathway 22. Actuation of the rotating component 86 may be manual and/or automated (e.g., initiated by a motor and/or controller).

To facilitate maintaining a fluid tight seal along the fluid pathway 22, a driven shaft 88 coupled with the rotating component 86 may extend from the rotating component 86 out of the cartridge 12 and couple with the drive shaft 80 extending from the motor 34 located in the reader device 14. When so configured, the drive components configured to actuate the rotating component 86 of the pump 68 may be fluidly isolated from the fluid pathway 22. In alternative configurations, the motor 34 may be located in the cartridge 12 and the drive shaft 80 may couple directly to the rotating component 86 and/or couple to the driven shaft 88.

The driven shaft 88 and the drive shaft 80 may be coupled in any suitable manner. In some examples, the driven shaft 88 and the drive shaft 80 may be mechanically coupled to one another via a keyed relationship, where one of the driven shaft 88 and the drive shaft 80 may receive the other of the driven shaft 88 and the drive shaft 80. Further, the driven shaft 88 may couple with the drive shaft 80 in any suitable manner including, but not limited to, via a magnetic connection. Other suitable configurations are contemplated.

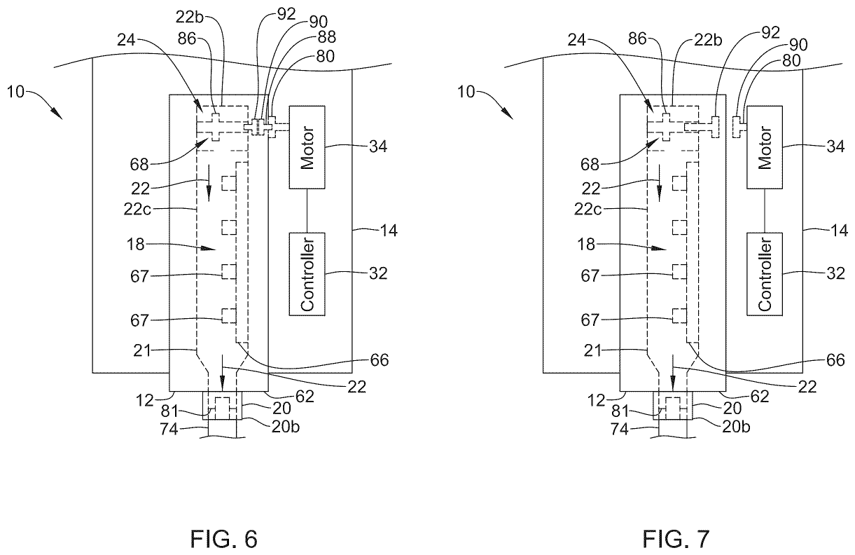

FIG. 6 schematically depicts an illustrative configuration of the system 10 with a similar configuration of the pump 68 as is depicted FIGS. 5A and 5B, but where the driven shaft 88 of the cartridge 12 is configured to couple with the rotating component 86 via a magnetic field (e.g., the pump 68 has a magnetic drive system). In the example depicted in FIG. 6, the driven shaft 88 may be mechanically coupled with the drive shaft 80 in communication with the motor 34. Then, as the driven shaft 88 rotates in response to rotation of the drive shaft 80 as actuated by the motor 34 and/or the controller 32, the rotating component 86 may rotate with the driven shaft 88 even though the driven shaft 88 and the rotating component 86 may be fluidly isolated from one another and are not physically coupled. When a magnetic field is used to rotate the rotating component 86, the drive and/or driven components of the pump 68 may be fluidly isolated from the fluid pathway 22.

The magnetic field extending between the rotating component 86 and the drive shaft 80 may be formed in any suitable manner. In some examples, one or more magnets, wound wire coils, and/or one or more magnetic materials may be utilized to create the magnetic field. In one example the driven shaft 88 may include a magnet 90 and the rotating component 86 may include a magnetic material 92 such that the magnetic field may extend from the driven shaft 88 to the rotating component 86. Alternatively or additionally, the driven shaft 88 may include the magnetic material 92 and the rotating component 86 may include the magnet 90 or both of the driven shaft 88 and the rotating component 86 may be or may include magnets 90. In some examples, the magnetic field may be generated using wound wire coils in communication with an electrical source (e.g., the controller 32, which provide electrical signals to the cartridge through electrical contacts between the reader device 14 and the cartridge 12), where the magnet may be replaced with the wound wire coils and the magnetic material may be rotated in response to actuation of the wire coils with electricity. Other suitable configurations are contemplated.

An alternative configuration of the pump 68 depicted in FIG. 6 may include mechanically coupling the drive shaft 80 with the rotating component 86, where a magnetic field may extend between the drive shaft 80 and the driven shaft 88. When so configured, the driven shaft 88 may include a magnet 90 and the drive shaft 80 may include a magnetic material 92 such that the magnetic field may extend from the driven shaft 88 to the drive shaft 80. Alternatively or additionally, the driven shaft 88 may include the magnetic material 92 and the drive shaft 80 may include the magnet 90 or both of the driven shaft 88 and the drive shaft 80 may be or may include magnets. Other suitable configurations are contemplated.

FIG. 7 schematically depicts an illustrative configuration of the system 10 with a similar configuration of the pump 68 depicted FIGS. 5A and 5B, but where the driven shaft 88 of the cartridge 12 is omitted and a magnetic field may extend between the rotating component 86 and the drive shaft 80. In the example depicted in FIG. 7, as the drive shaft 80 rotates in response to actuation by the motor 34 and/or the controller 32, the rotating component 86 may rotate with the drive shaft 80 even though the drive shaft 80 and the rotating component 86 are not physically coupled. When a magnetic field is used to rotate the rotating component 86, the drive components (e.g., the motor 34, the drive shaft 80, etc.) may be fluidly isolated from the driven components (e.g., the rotating component 86, etc.) of the pump 68 and fluidly isolated from the fluid pathway 22.

The magnetic field extending between the rotating component 86 and the drive shaft 80 may be formed in any suitable manner. In some examples, one or more magnets, wound wire coils, and/or one or more magnetic materials may be utilized to create the magnetic field. In one example, the drive shaft 80 may include a magnet 90 or wound wire coils and the rotating component 86 may include a magnetic material 92. Alternatively or additionally, the drive shaft 80 may include the magnetic material 92 and the rotating component 86 may include the magnet 90 or both of the drive shaft 80 and the rotating component 86 may be or may include magnets.

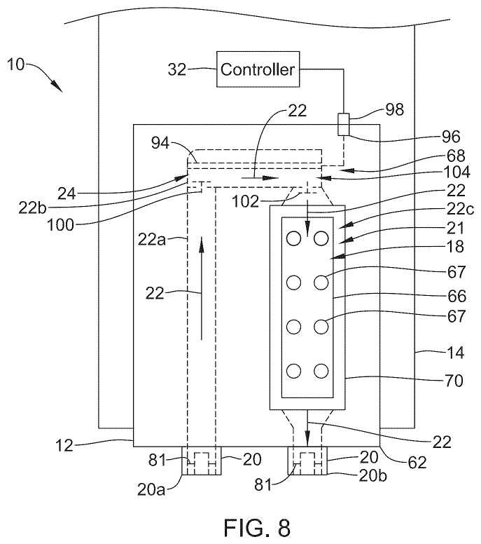

FIG. 8 schematically depicts a top view of an illustrative configuration of the system 10 with the cartridge 12 inserted into the reader device 14, where a pump component 24 of the cartridge 12 may define a portion of the the fluid pathway 22. The pump component 24 depicted in FIG. 8 may at least partially define the second portion 22b of the fluid pathway 22 and may be a piezoelectric diaphragm 94 of the pump 68 having a piezoelectric pump configuration. The configuration of the system 10 depicted in FIG. 8 may function similar to other configurations of the system 10 discussed herein, while using the illustrative configuration of the pump 68 and the fluid pathway 22 depicted in FIG. 8.

The piezoelectric diaphragm 94 may be formed in any suitable manner to facilitate pumping fluid through the fluid pathway 22. In some examples, the piezoelectric diaphragm 94 may be formed from a membrane with one or more piezoelectric materials or actuators coupled to the membrane, but other suitable configurations of the piezoelectric diaphragm 94 are contemplated.

The piezoelectric diaphragm 94 may be electrically coupled to a first contact 96 on the cartridge 12, which may be electrically coupled with a second contact 98 of the reader device 14 and in communication with the controller 32 when the cartridge 12 is inserted into the reader device 14. The first contact 96 and the second contact 98 may be mechanically coupled to one another to facilitate the electrical connection and/or positioning the cartridge 12 in the reader device 14, but other suitable configurations that do not include a mechanical connection between the first contact 96 and the second contact are contemplated.

The piezoelectric diaphragm 94 may at least partially define the second portion 22b of the fluid pathway 22, where the second portion 22b may extend from an inlet having a first valve 100 and an outlet having a second valve 102 and form a pump chamber 104. The first valve 100 and/or the second valve 102 may be any suitable type of valve including, but not limited to a valve plate, a gasket, a rubber valve, a ball and spring valve, plastic flaps, a single component configured to act as the first valve 100 and the second valve 102, and/or other suitable configurations. Other suitable configurations of the piezoelectric diaphragm 94, the fluid pathway 22, the valves 100, 102, and the pump chamber 104 are contemplated.

In operation, the piezoelectric diaphragm 94 may receive an electrical signal from the controller 32 to actuate the piezoelectric diaphragm 94. In some examples, the actuation of the piezoelectric diaphragm 94 may be configured to enlarge a volume of the pump chamber 104, which may open the first valve 100, close the second valve 102, and draw fluid into the pump chamber 104 through the inlet at the first valve 100. Then a further electrical signal from the controller 32 may be provided to the piezoelectric diaphragm 94 or the initial electrical signal may be removed to further actuate or relax the piezoelectric diaphragm 94 to reduce a volume of the pump chamber 104 relative to when the initial electrical signal is applied to the piezoelectric diaphragm 94. Reducing the volume of the pump chamber 104 may result in closing the first valve 100, opening the second valve 102, and causing fluid in the pump chamber 104 to leave the pump chamber 104 via the outlet at the second valve 102. Alternatively, in some examples an application of an initial electrical signal to the piezoelectric diaphragm 94 may be configured to reduce a volume of the pump chamber 104 to cause fluid to leave the pump chamber 104 via the outlet and removal of the initial electrical signal from and/or a further electrical signal to the piezoelectric diaphragm 94 may draw fluid into the pump chamber 104 via the inlet. Other suitable configurations are contemplated.

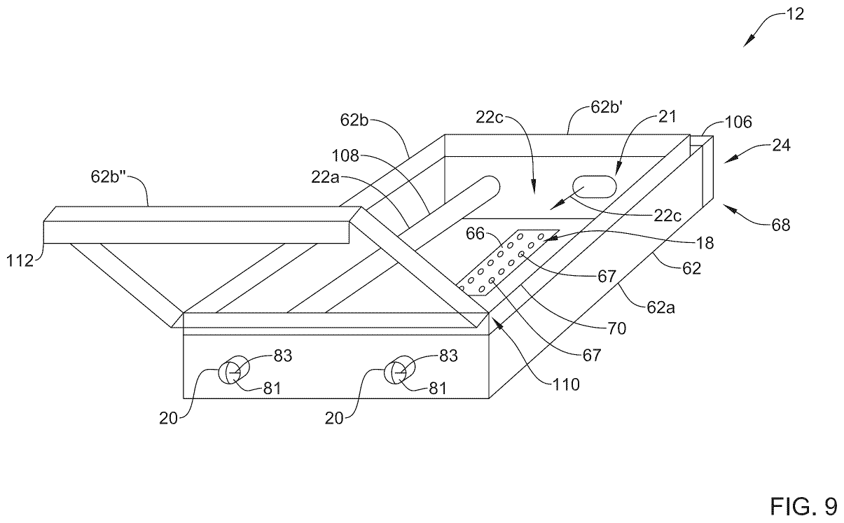

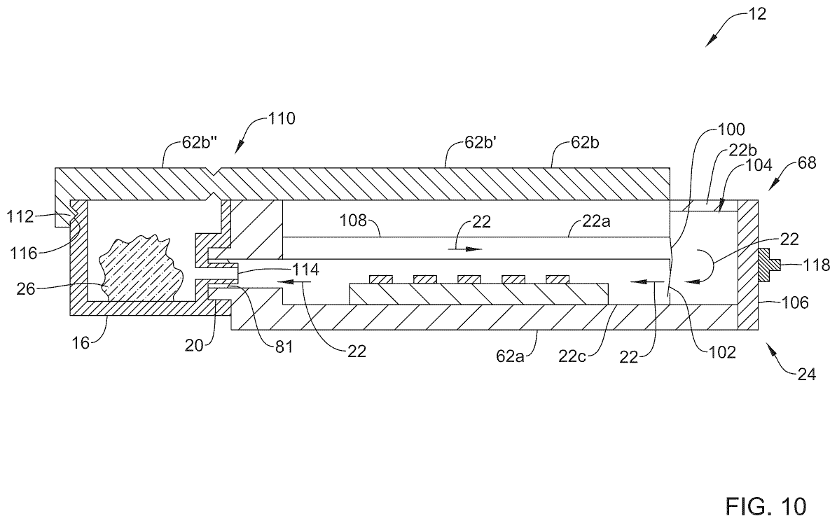

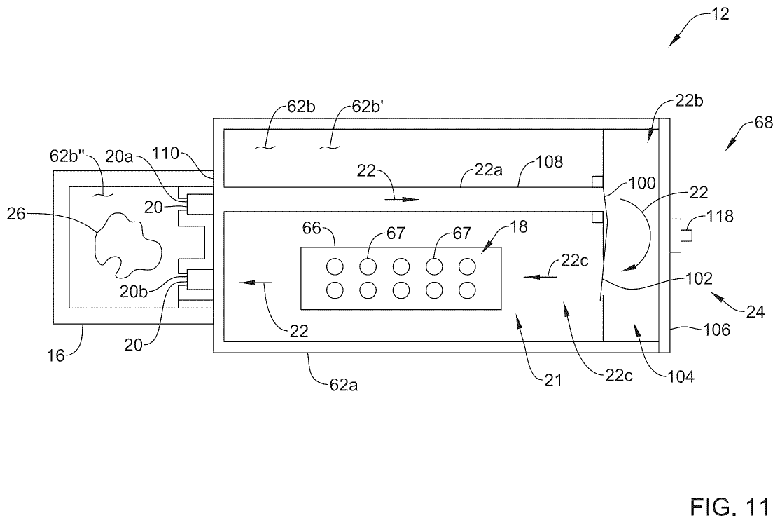

FIGS. 9-11 schematically depict portions an illustrative configuration of the cartridge 12, where a pump component 24 of the cartridge 12 may define a portion of the fluid pathway 22. The pump component 24 may at least partially define the second portion 22b of the fluid pathway 22 and may be a diaphragm 106 of the pump 68 (e.g., where the diaphragm 106 and a motor coupler 118 of the pump 68 are depicted in FIGS. 10 and 11 and the motor 34 and diaphragm coupler of the pump 68, which may be at the reader device 14, are not depicted). The configuration of the cartridge 12 depicted in FIGS. 9-11 may function similar to other configurations of the cartridge 12 discussed herein, while using the illustrative configuration of the pump 68 and the fluid pathway 22 depicted in FIGS. 9-11.

The diaphragm 106 and/or other suitable diaphragms discussed herein may be formed from any suitable material. Example suitable materials for diaphragms include, but are not limited to, metal materials, polymer materials, flexible materials, resilient materials, pliable materials, rigid materials, elastic materials, and/or other suitable materials.

The illustrative cartridge 12 depicted in FIGS. 9-11 may include the housing 62 having a first component 62a (e.g., a base) and a second component 62b (a lid or cover), the I/O ports 20, and the transparent portion 70 (e.g., an entirety or at least a portion of the second component 62b of the housing 62 formed from glass, polymer, and/or other suitable transparent material) through which the reader device 14 may view, project light onto, collect light from, and/or image the reactant array 18 positioned within the compartment 21 at least partially defining the fluid pathway 22 (e.g., the third portion 22c). The first component 62a of the housing 62 may entirely or at least partially define the compartment 21 in which the CSA 66 with the reactant array 18 is located and in which fluid from the sample source 16 may travel when the sample source 16 is in fluid communication with the compartment 21. Although the first portion 22a of fluid pathway 22 defined by a tube 108 is depicted as extending through the compartment 21, other configurations of the first portion 22a relative to the third portion 22c are contemplated.

The first component 62a of the housing 62 may include the I/O ports 20 configured to engage the sample source 16. For example, the I/O ports 20 may be configured to engage one or more components or fluidly coupled to the sample source 16 including, but not limited to, tubing, container(s) containing a sample (e.g., as depicted in FIGS. 10 and 11), containers configured to collect waste, and/or other suitable components. The engagement between the sample source 16 and the I/O ports 20 may be a releasable engagement or a permanent engagement.

The second component 62b may include a first portion 62b′ that may cover or act as a lid for the first component 62a of the housing 62 and a second portion 62b″ that is configured to engage and, optionally, act as a cover for the sample source 16 coupled with the first component 62a of the housing 62. The second portion 62b″ of the second component 62b may extend from the first portion 62b′ via a living hinge 110 or other suitable hinge and pivot relative to the first portion 62b′. In some configurations, the second portion 62b″ may include a latch 112 or other feature configured to engage the sample source 16 when the sample source 16 is coupled with the I/O ports 20.

One or more valves 81 may be positioned at or in the access openings extending through the I/O ports 20. In some examples, as depicted in FIG. 9, a single valve 81 (e.g., a check valve and/or other suitable type of valve) may be located in the access opening of each I/O port 20, where the single valve 81 may include a slit 83 or other opening biased closed and that may be opened in response to engagement with the sample source 16. When the ports of the sample source 16 extend through the valves 81, the valves 81 may be configured to seal around the ports.

The first portion 62b′ of the second component 62b of the housing 62 and the first component 62a may be fixedly sealed with respect to one another, but this is not required and the first component 62a and the second component 62b′ of the housing 62 may be adjustable with respect to one another to provide access to the compartment(s) 21 and/or the CSA 66. When the first component 62a and the second component 62b are sealed relative to one another, the seal may be a fluid-tight (e.g., hermetic) seal.

FIG. 9 schematically depicts a perspective view of the cartridge 12 with the second portion 62b″ of the second component 62b adjusted to an opened position to receive the sample source 16 and provide access to the access openings of the I/O ports 20. In the opened position, the second portion 62b″ may be rotated upward to facilitate the sample source 16 engaging the I/O ports 20. Alternatively, the cartridge 12 may include the sample source 16 (e.g., the sample source 16 may be part of the housing 62) and the second portion 62b″ arranged in the opened position may facilitate receiving the sample 26 in the sample source 16.

FIGS. 10 and 11 schematically depict a cross-section view and a top view of the configuration of the cartridge 12 depicted in FIG. 9, with the sample source 16 (e.g., in a container configuration) engaging the I/O ports 20 of the first component 62a of the housing 62. Although the sample source 16 is depicted as being connectable to the housing 62 and a separate component therefrom, the sample source 16 may be part of and/or a permanent structure of the housing 62, as discussed.

The sample source 16 may include one or more ports 114 configured to engage and/or extend through the I/O ports 20, as depicted in FIG. 10. In some examples, the ports 114 may be configured to extend through the valves 81 and the valves 81 may seal around the ports 114.

Before, during, or after the sample source 16 is in fluid communication with the I/O ports 20 and/or a sample 26 is in the sample source 16, the second portion 62b″ of the second component 62b may be adjusted and coupled with the sample source 16. In some examples, the second portion 62b″ may couple with the sample source 16 via a snap connection (e.g., a permanent connection or reversible connection) between the latch 112 and a notch 116 in the sample source 16. Although not required, the second portion 62b″ coupled with the sample source 16 may act as a cover to the sample 26 such that fluid from the sample 26 in the sample source 16 may only leave or enter the sample source 16 via the I/O ports 20, such that the cartridge 12 may form a closed-loop system 10 (e.g., a closed-loop fluid pathway 22). The second portion 62b″ of the second component 62b may form a fluid tight seal with the sample source 16, but other suitable configurations are contemplated.

Once the sample source 16 is securely coupled with the cartridge 12 and in fluid communication with fluid pathway 22, fluid may emanate from the sample 26 in the sample source 16, through the I/O port 20 coupled with the tube 108, through the tube 108 and the first portion 22a of the fluid pathway 22, into the pump chamber 104 and the second portion 22b of the fluid pathway 22, into the compartment 21 and the third portion 22c of the fluid pathway 22 through and/over the reactant array 18, through the I/O port 20 in fluid communication with the compartment 21, and into the sample source 16 to create the closed-loop fluid pathway 22. Alternatively, fluid may pass through the fluid pathway 22 in an opposite direction, such that fluid flows from the sample source 16, through the third portion 22c of the fluid pathway, through the second portion 22b of the fluid pathway 22, through the first portion 22a of the fluid pathway 22 and into the sample source 16. As the fluid passes to, through, and/or over the reactant array 18 of the CSA 66 in the compartment 21, the reactants 67 of the reactant array 18 may react to the fluid if an analyte of interest is detected. In some configurations, the fluid within the compartment 21 and/or the sample source 16 may be drawn from the sample source 16, through the fluid pathway 22, and back to the sample source 16 (or exhausted to one or more other suitable locations) via the pump 68.

The cartridge 12 may include, among other components of the pump 68, the diaphragm 106 and a motor coupler 118 (e.g., the diaphragm 106 and the motor coupler 118 may be pump components 24, where the motor coupler 118 is fluidly isolated from the fluid pathway 22). The diaphragm 106 may be a piezoelectric diaphragm, a diaphragm configured to respond to mechanical force, a diaphragm configured to respond to pressure forces, and/or other suitable diaphragm.

In operation, the motor coupler 118 may be configured to engage or couple (e.g., mechanically, electrically, magnetically, etc.) a motor, drive shaft, diaphragm coupler, piston, the controller, and/or other component of the pump 68 at the reader device 14 or at one or more other suitable locations such that the diaphragm 106 may be actuated to cause the first valve 100 to open, the second valve 102 to close, draw fluid through I/O port 20, into the tube 108, and through the inlet at the first valve 100 into the pump chamber 104, and then further act on the diaphragm 106 to close the first valve 100, open the second valve 102, push fluid into the compartment 21 through the outlet at the second valve 102, to, through, and/or over the reactant array 18, and out of the I/O port 20 in communication with the compartment 21. The configuration of the pump components 24 of the pump 68 at the cartridge 12 depicted in FIGS. 9-11 may be utilized with any suitable configurations of the fluid pathway 22, where the fluid pathway 22 has a closed loop configuration or an open loop configuration to circulate fluid through the fluid pathway 22.

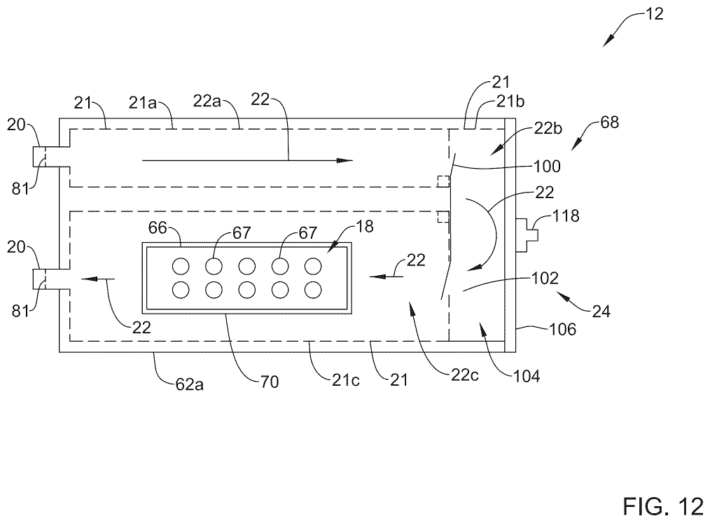

FIG. 12 schematically depicts a top view of an illustrative configuration of the cartridge 12, where a pump component 24 of the cartridge 12 may define a portion of the fluid pathway 22. The pump component 24 may at least partially define the second portion 22b of the fluid pathway 22 and may be a diaphragm 106 of the pump 68, but other suitable configurations are contemplated. The illustrative cartridge 12 depicted in FIG. 12 may include the housing 62 formed from one or more components, the one or more I/O ports 20, and the transparent portion 70 (e.g., a window and/or other suitable transparent portion formed from glass, polymer, and/or other suitable transparent material) through which the reader device 14 may view, collect light, and/or image the reactant array 18 positioned within a compartment 21.

The housing 62 may define any suitable number of compartments 21. For example, the housing 62 may define a first compartment 21a, a second compartment 21b, a third compartment 21c, and/or one or more other suitable compartments 21. As depicted in FIG. 12, the second compartment 21b may form the pump chamber 104 with the diaphragm 106. The CSA 66 with the reactant array 18 may be located in the first compartment 21a or the second compartment 21b (e.g., as depicted in FIG. 12).

The housing 62 may include the I/O ports 20 configured to engage the sample source 16. For example, and similar to as discussed with respect to FIGS. 9-11, the I/O ports 20 may be configured to engage one or more components of or fluidly coupled to the sample source 16 including, but not limited to, tubing, container(s) containing a sample (e.g., as depicted in FIGS. 10 and 11), containers configured to collect waste, and/or other suitable components. The engagement between the sample source 16 and the I/O ports 20 may be a releasable engagement or a permanent engagement.

The diaphragm 106 may function to pump fluid through the fluid pathway 22 in a manner similar to as discussed with respect to FIGS. 9-11. For example, the cartridge 12 may include, among other components of the pump 68, the diaphragm 106 and a motor coupler 118 (e.g., the diaphragm 106 and the motor coupler 118 may be pump components 24, where the motor coupler 118 may be fluidly isolated from the fluid pathway 22). The diaphragm 106 may be a piezoelectric diaphragm, a diaphragm configured to respond to mechanical force, a diaphragm configured to respond to pressure forces, and/or other suitable diaphragm.

In operation, the motor coupler 118 may engage or couple (e.g., mechanically, electrically, magnetically, etc.) with a motor, drive shaft, diaphragm coupler, piston, the controller, and/or other component of the pump 68 at the reader device 14 or at one or more other suitable locations such that the diaphragm 106 may be actuated to increase a volume of the pump chamber 104 and cause the first valve 100 to open, the second valve 102 to close, draw fluid through the I/O port 20 into the first compartment 21a along the first portion 22a of the fluid pathway 22, through the inlet at the first valve 100, and into the pump chamber 104, and then further actuate the diaphragm 106 to reduce a volume of the pump chamber 104 and close the first valve 100, open the second valve 102, push fluid through the outlet at the second valve 102, into the third compartment 21c, to, through, and/or over the reactant array 18, and through the I/O port 20 in communication with the third compartment 21c. The configuration of the pump components 24 of the pump 68 at the cartridge 12 depicted in FIG. 12 may be utilized with any suitable configurations of the fluid pathway 22, where the fluid pathway 22 has a closed loop configuration or an open loop configuration to circulate fluid through the cartridge 12.

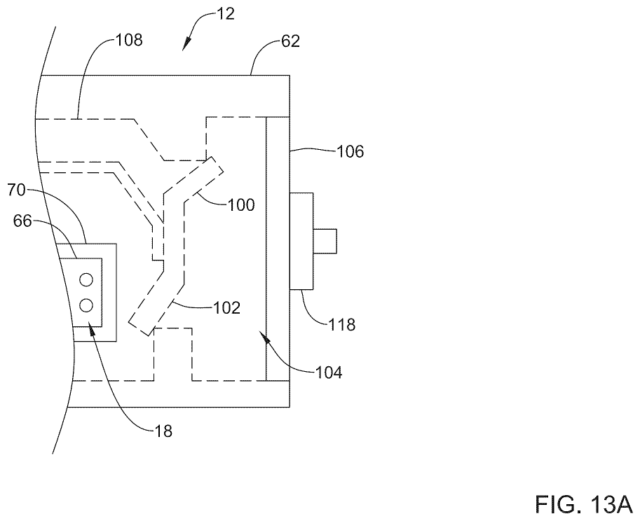

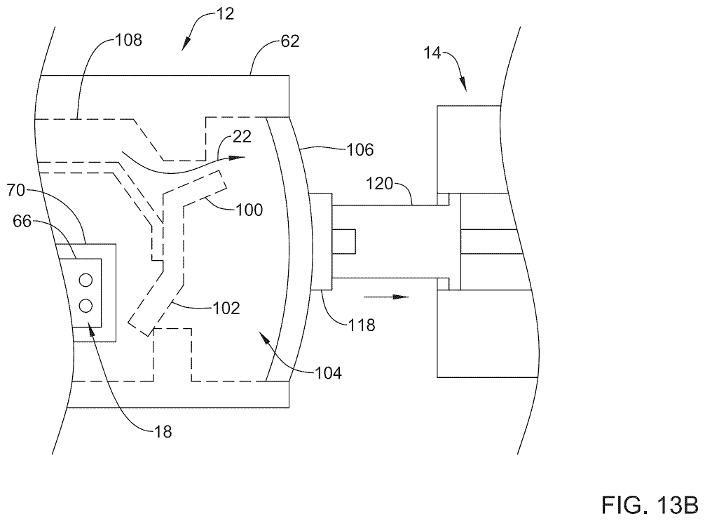

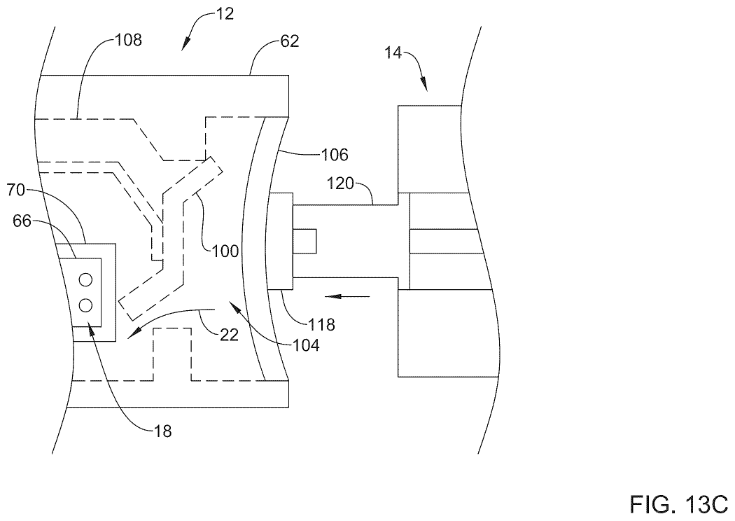

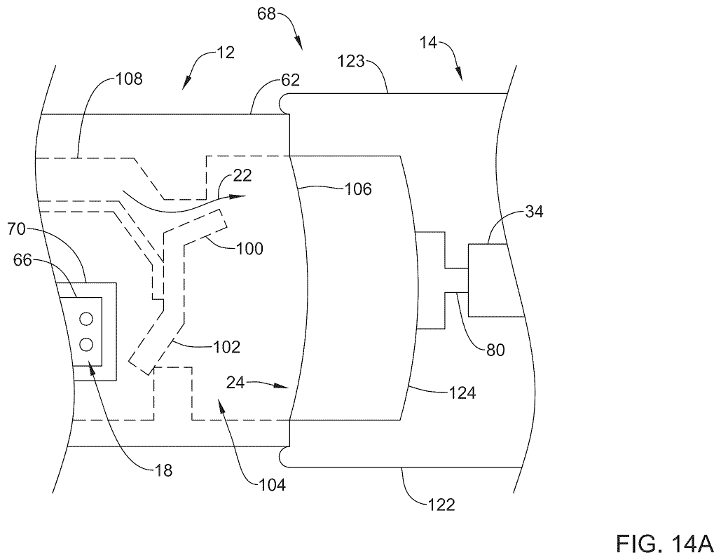

FIGS. 13A-13C schematically depict an illustrative technique for applying a mechanical force to the diaphragm 106 of the pump 68, where the diaphragm 106 may be located at the cartridge 12 of the system 10 and is actuated using a component fluidly isolated from fluid pathway 22. A diaphragm coupler 120 at the reader device 14 may be configured to couple with the motor coupler 118 to apply a mechanical force to the diaphragm 106 (e.g., to actuate the diaphragm 10) in response to a motor actuation or other suitable actuation.

FIG. 13A depicts a schematic view of a portion of the cartridge 12 including the pump chamber 104 at least partially defined by the diaphragm 106, where forces may act on the diaphragm 106 to open and close the first valve 100 and the second valve 102. Although the first valve 100 and the second valve 102 are depicted in FIG. 13A with an illustrative configuration, other suitable configurations the of valves with respect to the pump chamber 104 are contemplated, which may modify how movements of the diaphragm 106 affect fluid flow through the pump chamber 104.

FIG. 13B depicts the motor coupler 118 coupled with a diaphragm coupler 120 of the reader device 14, where the diaphragm coupler 120 may be in communication with a motor. In response to being actuated by the motor and in view of the configuration of the first valve 100 and the second valve 102, the diaphragm coupler 120 may apply a mechanical force to the diaphragm 106 through the motor coupler 118 to increase a volume of the pump chamber 104, open the first valve 100, close the second valve 102, and draw fluid into the pump chamber 104.

In response to a further actuation by the motor and in view of the configuration of the first valve 100 and the second valve 102, the diaphragm coupler 120 may apply a mechanical force to the diaphragm 106 through the motor coupler 118 to decrease a volume of the pump chamber 104, close the first valve 100, open the second valve 102, and push fluid out of the pump chamber 104 through the outlet at the second valve 102, as depicted in FIG. 13C. Additionally or alternatively, the diaphragm 106 may have or be formed from an elastic material or may be coupled to a bias, such that releasing an initial force acting on the diaphragm 106 to increase the volume of the pump chamber 104 may automatically reduce a volume of the pump chamber 104 and close the first valve 100, open the second valve 102, and push fluid out of the pump chamber 104 through the outlet at the second valve 102. In some examples, fluid exiting the pump chamber 104 may move to, through, and/or over the reactant array 18, as discussed herein or otherwise.

The stroke of the diaphragm 106 (e.g., a distance the diaphragm travels from a neutral position, such as depicted in FIG. 13A, to increase or decrease a volume of the pump chamber 104) and/or the rate of stroke cycles may be modified to control a flow volume and flow rate of fluid through the fluid pathway 22 and over or through the reactant array 18, where the stroke of the diaphragm 106 may determine a volume or pressure of fluid passing to, through, and/or over the reactant array 18 and the rate of stroke cycles may determine a flow rate of the fluid passing to, through, and/or over the reactant array 18. In some examples, if a first fluid will react better with the reactants of the reactant array 18 when the fluid is less pressurized at the reactant array 18 than is needed for a second fluid, a smaller stroke of the diaphragm 106 may be utilized for the first fluid than for the second fluid. In some examples, if it is known a first fluid takes longer to react with the reactant array 18 than a second fluid, the rate of stroke cycle may be set to a slower rate for the first fluid to reduce the flow rate relative to when the second fluid is being analyzed.

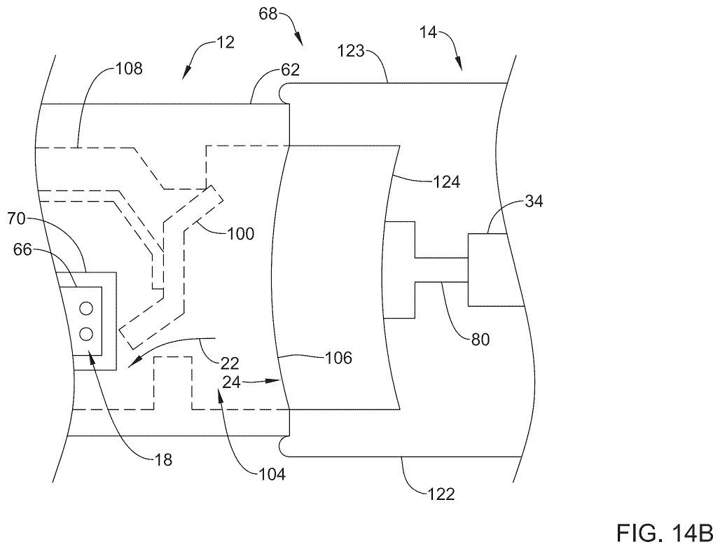

FIGS. 14A-14B schematically depict an illustrative technique for applying a pressurized force to the diaphragm 106 of the pump 68, where the diaphragm 106 may be located at the cartridge 12 of the system 10 and is actuated using a component fluidly isolated from fluid pathway 22. A cartridge coupler 122 at the reader device 14 may be configured to permanently or releasably couple with the housing 62 to apply a pressurized force to the diaphragm 106 in response to a motor actuation or other suitable actuation. Although the first valve 100 and the second valve 102 are depicted in FIGS. 14A and 14B with an illustrative configuration, other suitable configurations of the valves with respect to the pump chamber 104 are contemplated, which may modify how movements of the diaphragm affect fluid flow through the pump chamber 104.

The cartridge coupler 122 may have any suitable configuration configured to applying a pressurized force to the diaphragm 106 of the cartridge 12. In some examples, the cartridge coupler 122 may have a housing 123 configured to engage the housing 62 of the cartridge 12 and a diaphragm 124 or other suitable pressure changing component configured to adjust to changes in pressure applied to the diaphragm 106 of the cartridge 12. Further, the drive shaft 80 in communication with the motor 34 may be coupled to the diaphragm 124, such that actuation of the motor may result in moving or adjusting a position of the diaphragm 124 and changing a pressure applied to the diaphragm 106 of the cartridge 12.

FIG. 14A depicts the cartridge coupler 122 coupled with the housing 62 and the diaphragm 124 adjusted to a position that may reduce a pressure applied to the diaphragm 106 in response to actuation of the motor 34 and movement of the drive shaft 80. In response the diaphragm 124 being actuated by the motor 34 and in view of the configuration of the first valve 100 and the second valve 102, the reduced force (e.g., a negative or vacuum force) on the diaphragm 106 of the cartridge 12 may increase a volume of the pump chamber 104, open the first valve 100, close the second valve 102, and draw fluid into the pump chamber 104.

In response to a further actuation by the motor 34 and in view of the configuration of the first valve 100 and the second valve 102, the drive shaft 80 may be extended to cause the movement of diaphragm 124 and a pressurized force acting on the diaphragm 106 of the cartridge 12 to be increased. The increased pressurized force on the diaphragm 106 may decrease a volume of the pump chamber 104, close the first valve 100, open the second valve 102, and push fluid out of the pump chamber 104 through the outlet at the second valve 102, as depicted in FIG. 14B. Additionally or alternatively, the diaphragm 106 may have or may be formed from an elastic material or may be coupled to a bias, such that releasing the pressurized force acting on the diaphragm 106 to increase the volume of the pump chamber 104 may automatically reduce a volume of the pump chamber 104 and close the first valve 100, open the second valve 102, and push fluid out of the pump chamber 104 through the outlet at the second valve 102. In some examples, fluid exiting the pump chamber 104 may move to, through, and/or over the reactant array 18, as discussed herein or otherwise.

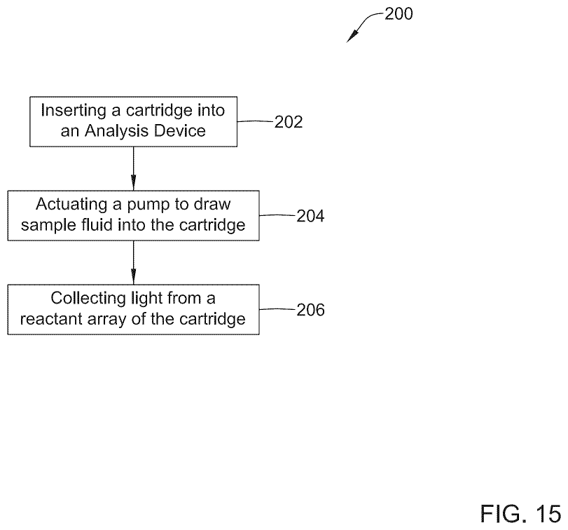

FIG. 15 schematically depicts an illustrative method 200 that may facilitate analyzing a reactant array (e.g., a reactant array of a CSA) exposed to a fluid from a sample (e.g., as part of a fluid analysis test on one or more fluids of interest). The method 200 may include inserting 202 a cartridge into a device (e.g., a reader device) configured to analyze reactant arrays. In some configurations, the cartridge may include a CSA including a reactant array configured to be exposed to a fluid and one or more ports configured to receive, engage, and/or be in fluid communication with a sample source including a sample or fluid from a sample to be tested. The device may be configured to collect light from the reactant array through the cartridge and analyze the collected light instantaneously and/or changes in the collected light over time.

Further, the method 200 may include actuating 204 a pump, at least partially implemented on the cartridge and, optionally, at least partially implemented on the reader device. Actuating the pump may draw fluid from the sample source into and/or through the cartridge along a fluid pathway, including through and/or over the reactant array, as discussed herein or otherwise. In some illustrative configurations, the pump may include a flexible, resilient, and/or pliable diaphragm, where actuating the pump may include applying a mechanical force, a pressurized force, and/or an electrical signal to the diaphragm to actuate the pump.

In some cases, the sample source may be positioned at or in fluid communication with the cartridge, where the reactant array of the CSA may be in a compartment of the cartridge. In some examples, the sample source may include a sample from which fluid may exude or be emitted to the reactant array. The sample may be collected from an area of interest by applying the specimen to the area of interest (e.g., a wound, pollen from a flower, an infection, an exhalation from a subject, a sweat gland, etc.)

In some configurations, once the cartridge is inserted into the reader device and/or the pump is actuated, the reader device may mark the cartridge as used, as discussed herein or otherwise. Further, the reader device that receives the cartridge may be able to detect if the cartridge has been previously used, as discussed herein or otherwise, and if the cartridge and/or specimen are determined to have been used, the reader device may reject performing a fluid analysis test using the specimen and/or cartridge.

Although not required, light from one or more light sources may be applied to the reactants of the reactant array. The light may be applied directly to the reactants of the reactant array and/or through a transparent portion of the substrate or cartridge. Application of light to the reactants of the reactant array may facilitate collecting light from the reactant array as the reactants are exposed to fluid.

The method 200 may include collecting 206 light from the reactant array of the CSA. The light from the reactant array may be collected with the reader device using a light or image sensor. In some examples, the light from the reactant array may be collected through a transparent portion of the cartridge. Although not required, the light may be collected from the reactant array while light is being applied to the reactant array.

In some configurations, the reader device may be configured to analyze levels of wavelengths of light collected from the reactant array. The levels of the wavelengths of light collected from the reactant array may be measured in any suitable manner including, but not limited to, by counting photons at one or more wavelengths of light collected, measuring an amount of light collected at one or more wavelengths of light collected, a change in photon count over time for one or more wavelengths of light collected, a change in pixel value (e.g., a change in pixel grayscale value) of an image sensor over time, and/or levels of wavelengths of light collected may be measured in one or more other suitable manners.

The measurements of the levels of the wavelengths of light collected from the reactant array may be utilized to identify a component of the fluid to which the reactant array may be exposed. In some examples, when the levels of the wavelengths of light collected over time match or closely resemble a set of expected wavelength levels of the reactant(s) exposed to a known fluid or component of fluid, the known fluid or component of fluid may be identified as the fluid or as a component of the fluid tested in the fluid analysis test. Example techniques for measuring levels of wavelength of light collected from reactant arrays and for comparing measurements to known measurements associated with fluids are discussed in U.S. Patent Application No. PCT/US2023/083024 (Attorney docket no. 1519.1004111), titled DEVICES, METHODS, AND SYSTEM FOR MEASURING AND RECORDING SPECTRUM OF A REACTANT ARRAY, having the same filing date as this application, which is hereby incorporated by reference in its entirety for any and all purposes.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Unless otherwise expressly stated, it is in no way intended that any method or technique set forth herein is to be construed as requiring that its steps be performed in a specific order. This holds for any possible non-express basis for interpretation, including matters of logic with respect to arrangement of steps or operational flow, plain meaning derived from grammatical organization or punctuation, and the number or type of embodiments described in the specification

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system comprising:
a cartridge, the cartridge includes a reactant array, an input port, an output port, and a pump component between the input port and the output port;
a device for analyzing the reactant array when the cartridge is received in the device, and
wherein the device is configured to engage the pump component to pump fluid through a pathway in fluid communication with the input port, the output port, and the reactant array.

2. The system of claim 1, wherein the pump component is fluidly isolated from the pathway.

3. The system of claim 1, wherein the device comprises an actuator configured to actuate the pump component.

4. The system of claim 1, wherein the pump component comprises an impeller.

5. The system of claim 1, wherein the pump component comprises a piezoelectric diaphragm configured to electrically connect with the device.

6. The system of claim 1, wherein the pump component comprises a flexible diaphragm at least partially defining the pathway.

7. The system of claim 6, wherein the device comprises a driving component configured to engage the flexible diaphragm to pump fluid through the pathway.

8. The system of claim 6, further comprising:
a first valve; and
a second valve, and
wherein the first valve is configured to open and the second valve is configured to close in response to a first movement of the flexible diaphragm and the first valve is configured to close and the second valve is configured to open in response to a second movement of the flexible diaphragm.

9. The system of claim 1, wherein the pathway is configured to be in fluid communication with a target area and the cartridge forms a closed loop system defining the pathway configured to be in fluid communication with the target area and causing fluid passing to the reactant array to return to the target area.

10. The system of claim 1, wherein the cartridge is a single-use component and the device is a reusable component.

11. The system of claim 1, wherein the cartridge comprises one or more seals configured to seal the input port and the output port to hermetically seal the reactant array within the cartridge.

12. The system of claim 1, wherein the device for analyzing the reactant array includes a pump drive component and the pump component of the cartridge is a pump driven component configured to be driven by the pump drive component when the cartridge is positioned within the device for analyzing the reactant array.

13. The system of claim 1, wherein:
the cartridge comprises a cartridge housing that houses the reactant array and the pump component, and
the device comprises a device housing configured to at least partially receive the cartridge housing.

14. A cartridge for use with a device for analyzing reactant arrays, the cartridge comprising:
a reactant array;
a pathway extending to the reactant array, the pathway is configured to receive fluid from a target area; and
a driven pump component configured to engage a drive pump component of the device for analyzing the reactant array to pump fluid through the pathway and to the reactant array.

15. The cartridge of claim 14, wherein the driven pump component is fluidly isolated from the pathway.

16. The cartridge of claim 14, wherein the driven pump component is selected from a group consisting of a flexible diaphragm, a rotor, a piezoelectric element, an Archimedes screw, and a plunger.

17. The cartridge of claim 14, further comprising:
a single-use component configured to prevent the reactant array from being reused.

18. The cartridge of claim 17, wherein the single-use component comprises one or more of the following types of single-use components: a mechanical single-use component, an electrical single-use component, an optical single-use component, a chemical single-use component, magnetic single-use component, and an electromechanical single-use component.

19. The cartridge of claim 14, further comprising:
a filter in the pathway, the filter is configured to remove undesirable molecules from the fluid.

20. The cartridge of claim 19, wherein:
the reactant array comprises a substrate and an array of reactants applied to the substrate, and
the substrate includes the filter.

21. A system comprising:
a cartridge, the cartridge includes a reactant array, an input port, an output port, and a flexible membrane, the cartridge includes a pathway configured to be in fluid communication with the input port, the output port, the reactant array, and the flexible membrane;
a device for analyzing the reactant array when the cartridge is received in the device; and
a pump configured to apply a force to the flexible membrane to pump fluid long the pathway and along the reactant array.

22. The system of claim 21, wherein the cartridge comprises a component of the pump.
